(19) United States
(12) Patent Application Publication
(10) Pub. No.: US 2009/0214644 A1
(43) Pub. Date: Aug. 27, 2009

(54) TRANEXAMIC ACID FORMULATIONS WITH REDUCED ADVERSE EFFECTS

(75) Inventors: Ralph A. HEASLEY, Webster Grove, MO (US); Keith A. Moore, Loveland, OH (US); Jeffrey S. Greiwe, Ft. Thomas, KY (US); John W. Facemire, Douglasville, GA (US); Jason D. Modest, Minneapolis, MN (US)

Correspondence Address: Davidson, Davidson & Kappel, LLC, 485 7th Avenue, 14th Floor, New York, NY, 10018, US

(73) Assignee: Xanodyne Pharmaceuticals, Inc., Newport, KY (US)

(21) Appl. No.: 12/433,247

(22) Filed: Apr. 30, 2009

Related U.S. Application Data
(63) Continuation-in-part of application No. 11/346,710, filed on Feb. 3, 2006.
Application No. 11/346,710 is a continuation-in-part of application No. 10/631,371, filed on Jul. 31, 2003, now abandoned.

(51) Int. Cl. A61K 31/195; A61K 9/22; A61P 43/00

(52) U.S. Cl. 424/468; 514/567

(57) ABSTRACT

Disclosed are delayed release oral tranexamic acid formulations and methods of treatment therewith.

This application is a continuation-in-part application of U.S. patent application Ser. No. 11/346,710, filed Feb. 3, 2006, which is a continuation-in-part application of U.S. patent application Ser. No. 10/631,371, filed Jul. 31, 2003, the disclosures of which are hereby incorporated by reference in its entirety.

### FIELD OF THE INVENTION

The invention is directed to therapeutic oral tranexamic acid formulations that minimize or eliminate undesirable side effects.

### BACKGROUND

Tranexamic acid (trans-4-(aminomethyl)cyclohexanecarboxylic acid, Cyklokapron® (Pfizer) is an antifibrinolytic agent. That is, it helps to prevent lysis or dissolution of a fibrin clot which forms in the normal physiologic process of hemostasis. Its mechanism of action is as a competitive inhibitor of plasminogen activation, and as a noncompetitive inhibitor of plasmin; both plasminogen and plasmin are activators of fibrinolyis and active clot-lysing agents. Tranexamic acid thus helps to stabilize fibrin clots, which in turn maintains coagulation and helps to control bleeding.

Tranexamic acid is used to control excess bleeding, for example, excess bleeding that occurs during dental procedures in hemophiliacs and for heavy bleeding during menstruation (menorrhagia). Women suffering from menorrhagia are typically treated orally with 500 mg tranexamic acid tablets administered three or four times daily with a total daily dose ranging from 3 grams/day (two tablets every eight hours) to 6 grams/day (three tablets every six hours). However, this treatment may cause adverse gastrointestinal reactions, including nausea, vomiting, diarrhea, and cramping, etc. These gastrointestinal side effects are due to the quantity of tranexamic acid introduced into the stomach with each dose, as well as the large quantity of excipients used in tablet formulation that are introduced into the stomach. Such side effects, in addition to the cramping, bloating, pain, and other symptoms that may accompany menses, are undesirable, and a formulation of tranexamic acid is needed which will reduce or eliminate these side effects.

### Menstrual Bleeding

Menstrual Bleeding disorders encompass a number of conditions including bleeding associated with uterine fibroids, endometriosis, or bleeding as a result of deficiencies in the clotting process for example, von-Willebrand's disease. Studies suggest that as many as 11% of the women who experience heavy menstrual bleeding, suffer from an inherited bleeding disorder such as von Willebrand's disease. Excessive Menstrual Bleeding is menstruation at relatively regular intervals but with excessive blood loss over the menses period which may be prolonged. Heavy Menstrual Bleeding (also referred to as “Menorrhagia”) is a serious, persistent, and recurrent medical condition that is one of the most common complaints encountered by gynecologists and primary care physicians (Palep-Singh, 2007). A 2005 survey of 273 obstetrician/gynecologists found that they see an average of 18 to 25 symptomatic patients per month. Heavy Menstrual Bleeding is a hyperfibrinolytic condition defined as cyclic, normal intervals of menstruation with excessive volume. Menorrhagia is often associated with a disruption in daily routines, work, and sexual activity leading to a significant decrease in health-related quality of life and time lost from work or school. While Menorrhagia is rarely life threatening, when undiagnosed and untreated, it may over time cause iron deficiency anemia and increased fatigue, both of which affect normal life activities, relationships, social activities, and various aspects of mental well-being (irritation, anxiety). Left untreated it may be associated with subsequent morbidity including dysmenorrhea, hospitalization, red blood cell transfusions and chronic pain. Annually, approximately 10% of women of reproductive age report Menorrhagia (Rees 1991; van Eijkeren, 1992) and according to the Center for Disease Control (CDC), 3 million women of reproductive age report Menorrhagia yearly, 60% of which have no known etiology. Studies report that as many as thirty percent of premenopausal women perceive their menses to be excessive.

Women suffering from menorrhagia often have greater uterine fibrinolytic activity than women with normal cyclic menstrual blood loss (MBL). High concentrations of plasminogen activators are found in both the uterus and menstrual fluid (Albrechtsen, 1956a,b). Rybo (1966) found significantly higher concentration of endometrial plasminogen activators in women with excessive menstrual bleeding compared to women with normal menstrual loss.

Causes of Menorrhagia include pelvic diseases (myomata [fibroids], adenomyosis or uterine polyps), intrauterine contraceptive devices, and systemic disorders (coagulopathies such as thrombocytopenia or von Willebrand's disease, and hypothyroidism). In contrast to menorrhagia, the term ‘dysfunctional uterine bleeding’ refers to excessive, prolonged or irregular bleeding from the endometrium that is unrelated to systemic disease (Wathen, 1995), and is usually associated with anovulation. Menorrhagia is also distinguished from other ovulatory bleeding disorders, such as metrorrhagia (intermenstrual bleeding), menometrorrhagia (irregular heavy menstrual bleeding) and polymenorrhea (menstrual cycle less than 21 days).

### Diagnosis of Menstrual Blood Loss

In clinical trials, menstrual blood loss (MBL) is usually determined by measuring the amount of hemoglobin recovered from sanitary products during the menstrual cycle, using the alkaline hematin method (Fraser, 1994). However, it is important to remember that blood accounts for only about 50% of total menstrual flow, with endometrial transudate accounting for the remainder (Fraser, 1994). Total menstrual flow can be estimated by weighing of sanitary products or by comparisons with a pictorial blood loss assessment chart. However, the use of these quantitative and semi-quantitative methods is not practical in non-trial settings. Rather, the diagnosis of Menorrhagia in the healthcare clinic is made by medical providers on the basis of patient's perceived and self-reported medical history, routine laboratory assessments of the patient's general health status, and gynecological examinations.

Clinically heavy menstrual bleeding is sometimes defined as total blood loss exceeding about 80 ml per cycle or menses lasting longer than seven days. The volume lost however, varies widely. Clinically losses from about 30 ml to 60 ml, 60 to 80 ml, 80 to 100 ml, to as high as 1000 ml per cycle are observed. Menstrual blood losses of 50 to 60 ml are associated with a negative iron balance and iron deficiency anemia is diagnosed in about 67% of the women who lose an excess of 80 ml per day. Other criteria for diagnosing the condition include measuring the number and size of blood clots in the meneges, or monitoring the use of pads or tampons. It is estimated that perhaps only ten percent of women who perceive their loss to be excessive actually fall within the clinical definition. The 80 ml definition has been repeatedly questioned, and alternative definitions broadened the blood loss range used for patient evaluations.

Blood loss volume assessments commonly require the collection and preservation of menstrual pads or tampons, the extraction of the pads and the accurate measurement of the blood content. Women are instructed to collect all sanitary towels and tampons during the course of the menstrual diagnosis period or the course of a clinical study period. Blood loss can be measured by extraction of the blood from the sanitary material with 5% sodium hydroxide followed with a spectrophotometric measurement of hematin at a wavelength of about 540 nm. The total blood loss can be calculated for an individual by comparison of the patients plasma blood hemoglobin measurement with the collected hemoglobin values.

The collection of the blood sample discourages the routine use of the test in the diagnosis or in the treatment of the condition. In the course of a routine visit with a physician other blood work may be appropriate but lacks a casual relation to the heavy bleeding disorder. The battery of routine laboratory tests may include patient blood hemoglobin, haematocrit, platelet count, bilirubin, serum creatinine and serum ferritin. In sum, diagnosis in the routine course of practice relies heavily on the woman's perception of the volume of blood lost during menses.

### Diagnosis and Treatment of Heavy Menstrual Bleeding Disorders (Menorrhagia)

A number of medical and surgical interventions are available to treat menstrual bleeding disorders. Currently available non-surgical treatments for heavy bleeding disorders, include, hormonal treatments (e.g., oral contraceptives), high-dose progestin therapy, desmopressin acetate, ethamsylate, nonsteroidal anti-inflammatory drugs (NSAIDs), the antifibrinolytic drugs aminocaproic acid and tranexamic acid. Even with the drug treatments available, surgery remains a common treatment.

Although not approved for menorrhagia in the US, use of oral contraceptives for menorrhagia is widely accepted. Oral contraceptives may not be a preferred therapy for some women because of age (younger females), unwanted side effects (nausea and vomiting, breakthrough bleeding, weight change, migraines and depression), and safety concerns (increased risk of thromboembolism, stroke, myocardial infarction, hepatic neoplasia and gall bladder disease). High-dose progestin (synthetic versions of the hormone progesterone) may also be given to women with menorrhagia, either orally or by a progestin-releasing device inserted into the uterus (intrauterine device). Side effects include nausea, bloating, mood changes, and breast tenderness.

Although it is typically a last resort, desmopressin acetate is sometimes used to help lighten menstrual flow in women with menorrhagia. The effectiveness of desmopressin is thought to vary between individuals. Side effects include headache, tachycardia, facial flushing, and rare reports of thromboembolism.

NSAIDs are sometimes used to treat menorrhagia as they may reduce blood flow while providing analgesia for pain associated with the condition (Shaw, 1994). Side effects associated with chronic NSAID use include gastrointestinal bleeding, ulceration, and perforation; and renal effects such as hyperkalemia, hyponatremia, acute renal insufficiency, interstitial nephritis, and renal papillary necrosis.

Hysterectomy or endometrial resection are options if other forms of therapy are not effective or are unsuitable for some reason. Possible surgical complications include infection, uterine perforation, and other complications associated with major surgery.

Antifibrinolytic drugs, such as ε-aminocaproic acid and tranexamic acid (immediate-release formulation) have been used to treat HMB in women with or without a diagnosed bleeding disorder (van Eijkeren, 1992; Bonnar, 1996; Vermylen, 1968; Nilsson, 1965). The available evidence from published literature suggests that tranexamic acid at doses of ˜4 g/day (typically 1 g every 6 hours) is effective in the treatment of HMB and is associated with few side effects (Callender, 1970; Dunn, 1999; Edlund, 1995; Preston, 1995). In Sweden, the average dose of tranexamic acid to treat HMB is 3.9 g/day (Rybo, 1991). Thus, tranexamic acid is used extensively in Europe, Canada, Asia, Japan, Australia and New Zealand to treat menorrhagia, but is not approved for this indication in the US.

Tranexamic acid is a competitive inhibitor of plasminogen activation (see review by Dunn, 1999). Binding of tranexamic acid to plasminogen does not prevent conversion of plasminogen to plasmin by tissue plasminogen activator, but the resulting plasmin/tranexamic acid complex is unable to bind to fibrin. Thus, enzymatic breakdown of fibrin by plasmin (fibrinolysis) is inhibited. At higher concentrations, tranexamic acid is also a noncompetitive inhibitor of plasmin.

Before medical and surgical interventions can be initiated, diagnosis of a heavy menstrual bleeding disorder must be accomplished.

Diagnosis and treatment of disease often depends on the patient's perception and subsequent description of symptoms, the physician's evaluation of the patient's description, the physician observations of the patient and laboratory test results. Menstrual bleeding disorders do not lend themselves to physician observation or to routine laboratory testing. Patient observations and the physician's evaluation of the patient's description are subjective and thus variable. In addition a women's medical history has been found to be a poor predictor of menstrual blood loss. Neither the duration of menses nor the number of sanitary pads worn accurately corresponds to the woman's actual menstrual blood loss (Chimbira, Haynes, year). An objective assessment of blood loss using the alkaline haematin assay has been shown to be reproducible but it is not suited for routine clinical use by healthcare providers. To date no effective instrument for reliably diagnosing and/or monitoring the treatment of menstrual bleeding disorders has been developed despite the significant number of women who suffer from these conditions.

Previously, studies have focused on the impact of symptoms of bleeding disorders on patients' health related quality of life. As the effects of menstrual bleeding disorders are primarily symptomatic, the subjective outcome namely symptom alleviation, cannot be objectively measured. In research from European countries where the antifibrinolytic drug tranexamic acid is currently available, treatment with this antifibrinolytic has reduced heavy menstrual bleeding by 40-50% and improved the health-related quality of life of affected women on measures of social activity, work performance, productivity, cleanliness, overall functioning and tiredness.

Jenkinson et al, Quality in Health Care 1996; 5; 9-12 evaluated the validity and internal reliability of the short form-36 (SF36) health survey questionnaire in women presenting with menorrhagia. The study concluded that several questions on the questionnaire were difficult to answer for patients with heavy menstrual bleeding. Such problems were suggested as possible interferences with the validity of the measure. Jenkinson warns that because a subjective measure works well in one population or with one group, this cannot be taken to imply its appropriateness for all groups or conditions.

Edlund, in an abstract from a seminar on Dysfunctional Uterine Bleeding, Feb. 23, 1994, indicates that a questionnaire was used in a Swedish study of 2205 women who described their menstruation as excessive.

Winkler in a study based in part on the Edlund work, concluded that the treatment of heavy menstrual bleeding with tranexamic acid increased the quality of life of the treated patients. The Winkler study was an open label uncontrolled usage study which included 849 patients. A questionnaire was used prior to treatment and after the first and third menstruation. The study indicates that 80% of the women were satisfied with the treatment. The questionnaire used a series of eight question combined with an assessment by the patients of the change in quantity of menstrual flow.

Ruta, D. A., Quality of Life Research, 4, (33-40), 1995 finds that menorrhagia is a common problem in gynecological practice and that women seek professional help primarily because of the deleterious effect on their quality of life. Ruta recognizing the importance of evaluating the effectiveness of the treatments developed a questionnaire based on the type of questions frequently asked when taking a gynecological history. A series of questions were devised which assessed fifteen factors including the duration of the period, the regularity of the period, pain, problems with soiling/staining, interference with work, interference with leisure. Ruta concluded that the clinical questionnaire may be useful in selecting patients for hysterectomy and assessing the outcome of conservative treatment especially in combination with the SF-36 questionnaire.

### Diagnostic Test for Menstrual Bleeding

The alkaline haematine test described above provides quantitative assessments of the extent of menstrual bleeding. This test allows the physician to diagnose and monitor the progress of a women's menstrual process. However the test is impractical and difficult to perform. The test requires women to capture used menstrual pads over the course of her period, preserve the samples in a condition such that the blood content within the pad may be accurately extracted and quantitated. Requesting a patient to perform menses sample collection may be practical in the course of a clinical trial where procedures are specified and monitored however, in routine medical practice, the use of such a test procedure to diagnose and monitor a women's menstrual bleeding is impractical and the data generated is unreliable.

The need remains to develop an assessment system which replaces previously studied diagnostic techniques and the alkaline haematine test and provides a reliable measure of both the occurrence of the disorder and the progress of the disorder. The present invention fills this need by providing a Heavy Menstrual Bleeding Instrument (HMBI) which is capable of diagnosing, and monitoring the treatment of a patient with a menstrual bleeding disorder.

There also remains a need to provide Heavy Menstrual Bleeding (HMB) therapy that is safe, efficacious and only administered during the monthly period of heavy menstruation, addresses the excessive fibrinolysis implicated in many causes of menorrhagia, and fills a currently recognized unmet medical need in the US. Therapy for HMB is expected to reduce the incidence and extent of iron-deficiency anemia, and to provide a nonhormonal medical therapy option in lieu of the numerous invasive procedures (e.g., transcervical endometrial resection) and major surgery (hysterectomy) performed annually.

### SUMMARY OF THE INVENTION

Formulations of tranexamic acid which minimize or eliminate the undesirable gastrointestinal side effects in patients on oral tranexamic acid therapy, e.g. women treated for menorrhagia (heavy menstrual bleeding), by modifying the release characteristics of tranexamic acid are disclosed. One embodiment is an extended release formulation, also termed a controlled release formulation, formulated so that the release of tranexamic acid from the dosage form occurs in an extended or controlled fashion to prevent a bolus of tranexamic acid being introduced into the stomach and available for dissolution in the gastric contents. An alternative embodiment is a delayed release formulation. Delayed release dosage forms are formulated to minimize or prevent the dissolution of the drug in the stomach. The release of tranexamic acid is delayed until the dosage form exits the stomach and reaches the small intestine. Both extended release dosage forms and delayed release dosage forms are termed modified release dosage forms. Such modified release formulations reduce the concentration of tranexamic acid dissolved in the stomach contents. The beneficial effect of this reduced tranexamic acid concentration is to lower the amount of tranexamic acid in the gastric contents so that there are fewer gastric adverse effects with tranexamic acid therapy. This reduction in gastric adverse effects results in improved patient compliance with therapy, because patients will not intentionally miss taking a dose to avoid these adverse side effects. Physicians will also be more likely to initiate and maintain tranexamic acid treatment for their patients because of the reduced patient complaints.

It is an object of the invention to provide an oral dosage form comprising tranexamic acid which is suitable for administration on a two or three times a day basis to humans.

It is an object of the invention to provide a modified release oral dosage form comprising tranexamic acid and a modified release material which provides for the modified release of the tranexamic acid and is suitable for administration on a two or three times a day basis.

It is an object of the invention to provide a delayed release oral dosage form comprising tranexamic acid and a delayed release material which provides for the delayed release of the tranexamic acid and is suitable for administration on a two or three times a day basis.

It is a further object of certain embodiments of the present invention to provide a modified release oral dosage form comprising tranexamic acid and a modified release material which minimizes or eliminates the undesirable gastrointestinal side effects in patients on oral tranexamic acid therapy while maintaining or improving the therapeutic effect of tranexamic acid.

It is a further object of certain embodiments of the present invention to provide a delayed release oral dosage form comprising tranexamic acid and a delayed release material which minimizes or eliminates the undesirable gastrointestinal side effects in patients on oral tranexamic acid therapy while maintaining or improving the therapeutic effect of tranexamic acid.

It is a further object of certain embodiments of the present invention to provide a method of treating a patient suffering from heavy menstrual bleeding (menorrhagia) by orally administering to the patient one or more dosage forms comprising tranexamic acid and a modified release material which provide(s) for therapeutically effective levels of tranexamic acid suitable for two or three times a day administration.

It is a further object of certain embodiments of the present invention to provide a method of treating a patient suffering from heavy menstrual bleeding (menorrhagia) by orally administering to the patient one or more dosage forms comprising tranexamic acid and a delayed release material which provide(s) for therapeutically effective levels of tranexamic acid suitable for two or three times a day administration.

The above advantages and objects and others can be achieved by virtue of the present invention which is directed in part to a modified release oral dosage form comprising tranexamic acid and a modified release material which provides for the modified release of the tranexamic acid from the dosage form such that the dosage form is suitable for administration on a two or three times a day basis.

In certain embodiments, the present invention is further directed to a delayed release oral dosage form comprising tranexamic acid and a delayed release material which provides for the delayed release of the tranexamic acid from the dosage form such that the dosage form is suitable for administration on a two or three times a day basis.

In certain embodiments, the present invention is directed to a delayed release oral dosage form comprising tranexamic acid or a pharmaceutically acceptable salt thereof and a delayed release material which provides for the delayed release of the tranexamic acid such that the dosage form is suitable for administration on a two or three times a day basis; said dosage form providing an in-vitro dissolution release rate of the tranexamic acid or pharmaceutically acceptable salt thereof when measured by the USP 27 Dissolution Apparatus Type II Paddle Method @ 50 RPM and 37±0.5° C. of less than about 10% by weight tranexamic acid or pharmaceutically acceptable salt thereof released by about 120 minutes in acid medium (1000 ml of 0.1N hydrochloric acid), and at least about 75% by weight of said tranexamic acid or pharmaceutically acceptable salt thereof released by about 45 minutes after subsequent immersion in buffer medium (1000 ml of pH 6.8 phosphate buffer), preferably less than about 5% by weight tranexamic acid or pharmaceutically acceptable salt thereof released by about 120 minutes in the acid medium and at least about 90% by weight tranexamic acid or pharmaceutically acceptable salt thereof released by about 45 minutes after subsequent immersion in the buffer medium.

In certain embodiments, the present invention is further directed to a modified release oral dosage form comprising from about 585 to about 715 mg of tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a modified release material which provides for the modified release of the tranexamic acid or pharmaceutically acceptable salt thereof from the dosage form such that the dosage form is suitable for administration on a two or three times a day basis.

In certain embodiments, the present invention is further directed to a delayed release oral dosage form comprising from about 585 to about 715 mg of tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a delayed release material which provides for the delayed release of the tranexamic acid or pharmaceutically acceptable salt thereof from the dosage form such that the dosage form is suitable for administration on a two or three times a day basis.

In certain embodiments, the present invention is directed to a modified release oral dosage form comprising tranexamic acid or pharmaceutically acceptable salt thereof and a modified release material which provides for the modified release of the tranexamic acid or pharmaceutically acceptable salt thereof from the dosage form such that the dosage form is suitable for administration on a two or three times a day basis, the dosage form providing a reduction of at least one side effect selected from the group consisting of headache, nausea, vomiting, diarrhea, constipation, cramping, bloating, and combinations thereof, as compared to an equivalent amount of tranexamic acid or pharmaceutically acceptable salt thereof in an immediate release oral dosage form when administered across a patient population.

In certain embodiments, the present invention is directed to a delayed release oral dosage form comprising tranexamic acid or pharmaceutically acceptable salt thereof and a delayed release material which provides for the delayed release of the tranexamic acid or pharmaceutically acceptable salt thereof from the dosage form such that the dosage form is suitable for administration on a two or three times a day basis, the dosage form providing a reduction of at least one side effect selected from the group consisting of headache, nausea, and combination thereof, as compared to an equivalent amount of tranexamic acid or pharmaceutically acceptable salt thereof in an immediate release oral dosage form when administered across a patient population.

In certain embodiments, the invention is further directed to a method of treating a patient with a therapeutically effective amount of tranexamic acid or pharmaceutically acceptable salt thereof comprising administering to the patient two dosage forms of the present invention, each dosage form comprising from about 585 mg to about 715 mg of tranexamic acid or pharmaceutically acceptable salt thereof, preferably from about 617 to about 683 mg of tranexamic acid or pharmaceutically acceptable salt thereof, more preferably about 650 mg tranexamic acid or pharmaceutically acceptable salt thereof, and a modified release material such that the dosage form is suitable for oral administration on a three times a day basis.

In certain embodiments, the invention is further directed to a method of treating a patient with a therapeutically effective amount of tranexamic acid or pharmaceutically acceptable salt thereof comprising administering to the patient two dosage forms of the present invention, each dosage form comprising from about 585 mg to about 715 mg of tranexamic acid or pharmaceutically acceptable salt thereof, preferably from about 617 to about 683 mg of tranexamic acid or pharmaceutically acceptable salt thereof, more preferably about 650 mg tranexamic acid or pharmaceutically acceptable salt thereof, and a delayed release material such that the dosage form is suitable for oral administration on a three times a day basis.

In certain embodiments, the invention is further directed to a method of treating a patient with a therapeutically effective amount of tranexamic acid or pharmaceutically acceptable salt thereof comprising administering to the patient three dosage forms of the present invention, each dosage form comprising from about 585 mg to about 715 mg tranexamic acid or pharmaceutically acceptable salt thereof, preferably from about 617 to about 683 mg of tranexamic acid or pharmaceutically acceptable salt thereof, more preferably about 650 mg tranexamic acid or pharmaceutically acceptable salt thereof, and a modified release material such that the dosage form is suitable for oral administration on a twice a day basis.

In certain embodiments, the invention is further directed to a method of treating a patient with a therapeutically effective amount of tranexamic acid or pharmaceutically acceptable salt thereof comprising administering to the patient three dosage forms of the present invention, each dosage form comprising from about 585 mg to about 715 mg tranexamic acid or pharmaceutically acceptable salt thereof, preferably from about 617 to about 683 mg of tranexamic acid or pharmaceutically acceptable salt thereof, more preferably about 650 mg tranexamic acid or pharmaceutically acceptable salt thereof, and a delayed release material such that the dosage form is suitable for oral administration on a twice a day basis.

In certain embodiments, the invention is directed to a dose of tranexamic acid or pharmaceutically acceptable salt thereof comprising two unit dosage forms of a modified release formulation, each unit dosage form of said modified release formulation comprising from about 585 mg to about 715 mg of tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a modified release material which provides for the release of the tranexamic acid or pharmaceutically acceptable salt thereof from the dosage form such that the dose provides a therapeutic effect when administered three times a day.

In certain embodiments, the invention is directed to a dose of tranexamic acid or pharmaceutically acceptable salt thereof comprising two unit dosage forms of a delayed release formulation, each unit dosage form of said delayed release formulation comprising from about 585 mg to about 715 mg tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a delayed release material which provides for the release of the tranexamic acid or pharmaceutically acceptable salt thereof from the dosage form such that the dose provides a therapeutic effect when administered three times a day.

In certain embodiments, the invention is directed to a dose of tranexamic acid comprising three unit dosage forms of a modified release formulation, each unit dosage form of said modified release formulation comprising from about 585 mg to about 715 mg tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a modified release material which provides for the release of the tranexamic acid or pharmaceutically acceptable salt thereof from the dosage form such that the dose provides a therapeutic effect when administered twice a day.

In certain embodiments, the invention is directed to a dose of tranexamic acid comprising three unit dosage forms of a delayed release formulation, each unit dosage form of said modified release formulation comprising from about 585 mg to about 715 mg tranexamic acid or pharmaceutically acceptable salt thereof, preferably from about 617 to about 683 mg of tranexamic acid or pharmaceutically acceptable salt thereof, more preferably about 650 mg of tranexamic acid or pharmaceutically acceptable salt thereof, and a delayed release material which provides for the release of the tranexamic acid or pharmaceutically acceptable salt thereof from the dosage form such that the dose provides a therapeutic effect when administered twice a day.

In certain embodiments, the invention is further directed to a modified release oral dosage form comprising tranexamic acid or pharmaceutically acceptable salt thereof and a delayed release material which provides for the delayed release of the tranexamic acid or pharmaceutically acceptable salt thereof from the dosage form such that the dosage form is suitable for administration on a two or three times a day basis and the dosage form provides less than about 25 percent incidence of headache as a side effect after single dose oral administration across a patient population.

In certain embodiments, the invention is further directed to a delayed release oral dosage form comprising tranexamic acid or pharmaceutically acceptable salt thereof and a delayed release material which provides for the delayed release of the tranexamic acid or pharmaceutically acceptable salt thereof from the dosage form such that the dosage form is suitable for administration on a two or three times a day basis and the dosage form provides less than about 10 percent incidence of nausea as a side effect when administered across a patient population, preferably less than about 7 percent incidence of nausea when administered across a patient population, more preferably less than about 5 percent incidence of nausea as a side effect when administered across a patient population, most preferably less than about 2 percent incidence of nausea as a side effect after single dose oral administration across a patient population.

In certain embodiments, the delayed release oral dosage form of the present invention provides less CNS side effects (e.g., headache), less GI side effects (e.g., nausea), or combination thereof in comparision to an equivalent amount of tranexamic acid or pharmaceutically acceptable salt thereof in an immediate release formulation when administered across a patient population. Additionally or alternatively, in certain embodiments the dosage form provides less CNS side effects (e.g., headache), less GI side effects (e.g., nausea), or combination thereof in comparision to a therapeutically equivalent amount of tranexamic acid administered intravenously in five minutes or less across a patient population.

In certain embodiments, the delayed release oral dosage form of the present invention provides for the reduction of at least one side effect as compared to an immediate release oral dosage form including an equivalent amount of tranexamic acid or pharmaceutically acceptable salt thereof, when the immediate release dosage form is administered across a same or different population of patients as said delayed release dosage form, and wherein said immediate release dosage form releases all of said tranexamic acid or pharmaceutically acceptable salt thereof within about 45 minutes when measured in vitro utilizing the USP 27 Dissolution Apparatus Type II Paddle Method @ 50 RPM in 900 ml water at 37±0.5° C. Such side effects can be for example, headache, nausea, and combinations thereof.

In certain embodiments, the present invention is directed to a delayed release oral dosage form comprising tranexamic acid or a pharmaceutically acceptable salt thereof and a delayed release material which provides for the delayed release of the tranexamic acid such that substantially none of said tranexamic acid is released from said dosage form after exposure of said dosage form for a period of 2 hours to an environmental fluid having a pH of less than about 2, preferably less than about 3, and substantially all of said tranexamic acid is released from said dosage form after exposure of said dosage form for a period of 1 hour to an environmental fluid having a pH of at least about 5, preferably at least about 6, more preferably at least about 7.

In certain embodiments, the present invention is directed to a delayed release oral dosage form comprising tranexamic acid or a pharmaceutically acceptable salt thereof and a delayed release material which provides for the delayed release of the tranexamic acid such that less than about 10% by weight, preferably less than about 5% by weight of said tranexamic acid is released from said dosage form after exposure of said dosage form for a period of 2 hours to an environmental fluid having a pH of less than about 2, preferably less than about 3, and at least about 75% by weight, preferably at least about 95% by weight of said tranexamic acid is released from said dosage form after exposure of said dosage form for a period of 1 hour to an environmental fluid having a pH of at least about 5, preferably at least about 6, more preferably at least about 7.

In certain preferred embodiments, the therapeutically effective dose of the tranexamic acid or pharmaceutically acceptable salt thereof is provided via the administration of two or more dosage units. For example, if the dosage unit comprises 650 mg of tranexamic acid or pharmaceutically acceptable salt thereof and the dose for administration is about 1300 mg then two dosage units would be administered to a patient in need of such treatment, or for example, when the dose for administration is 1950 mg, three dosage units would be administered.

In certain preferred embodiments, the invention is further directed to a method of treating a patient with one or more delayed release oral dosage forms comprising tranexamic acid or pharmaceutically acceptable salt thereof and a delayed release material, wherein the oral dosage form provides a therapeutically effective plasma level of tranexamic acid or pharmaceutically acceptable salt thereof in accordance with a three times a day (TID) dosing schedule, and the therapeutically effective dose administered comprises about 1300 mg of tranexamic acid or pharmaceutically acceptable salt thereof.

In certain preferred embodiments, the invention is further directed to a method of treating a patient with one or more delayed release oral dosage forms comprising tranexamic acid or pharmaceutically acceptable salt thereof and a delayed release material, wherein the oral dosage form provides a therapeutically effective plasma level of tranexamic acid or pharmaceutically acceptable salt thereof in accordance with a twice a day (BID) dosing schedule, and the therapeutically effective dose administered comprises about 1950 mg of tranexamic acid or pharmaceutically acceptable salt thereof.

In certain embodiments, the invention is directed to a method of providing a tranexamic acid plasma concentration within the range of about 5 mcg/mL to about 15 mcg/mL by administration of a delayed release formulation of the present invention comprising tranexamic acid or pharmaceutically acceptable salt thereof and a delayed release material on a three times a day basis to a patient in need of tranexamic acid or pharmaceutically acceptable salt thereof treatment.

In certain embodiments, the invention is further directed to a method of treating a human patient with heavy menstrual bleeding (e.g., menorrhagia) comprising administering about 1300 mg of tranexamic acid or pharmaceutically acceptable salt thereof on a three times a day basis to the human patient to provide a tranexamic acid or pharmaceutically acceptable salt thereof plasma concentration within the range of about 5 mcg/mL to about 15 mcg/mL after steady state oral administration to a human patient.

In certain embodiments, the invention is directed to a method of treating a patient suffering from menorrhagia, including patients with heavy menstrual bleeding due to fibroids, conization of the cervix, epistaxis, hyphema, hereditary angioneurotic edema, a patient with a blood coagulation disorder undergoing dental surgery, combinations thereof, and the like, by administering at least one dosage form of the present invention to the patient in need in tranexamic acid or pharmaceutically acceptable salt thereof therapy.

In certain other embodiments, the present invention is directed to the use of the tranexamic acid formulations described herein for the treatment of heavy menstrual bleeding (menorrhagia) and the amelioration of symptoms associated with heavy menstrual bleeding, including limitations on social, leisure, and physical activities.

In certain embodiments, the modified release and/or delayed release material may be incorporated in a coating applied onto a tablet comprising the tranexamic acid or pharmaceutically acceptable salt thereof, or may be incorporated into a matrix with the tranexamic acid or pharmaceutically acceptable salt thereof, or a combination the two. For example, in certain preferred embodiments, the modified release material is a controlled release material such as a gel-forming or hydratable polymer which is added to a matrix composition comprising the tranexamic acid or pharmaceutically acceptable salt thereof.

In certain preferred embodiments, the delayed release oral dosage form comprises a core comprising tranexamic acid or a pharmaceutically acceptable salt thereof and a pharmaceutically acceptable excipient; and a delayed release material coated on the core which provides for the delayed release of the tranexamic acid or pharmaceutically acceptable salt thereof, such that the dosage form is suitable for administration on a two or three times a day basis.

In certain further preferred embodiments, the delayed release oral dosage form comprises a matrix comprising tranexamic acid or a pharmaceutically acceptable salt thereof and a delayed release material which provides for the delayed release of the tranexamic acid such that the dosage form is suitable for administration on a two or three times a day basis.

In certain preferred embodiments, the delayed release oral dosage form comprises a plurality of multiparticulates (e.g., inert beads, matrix multiparticulates, etc.) comprising tranexamic acid or a pharmaceutically acceptable salt thereof and a delayed release material which provides for the delayed release of the tranexamic acid or pharmaceutically acceptable salt thereof such that the dosage form is suitable for administration on a two or three times a day basis.

In certain embodiments, the tranexamic acid for use in the methods and formulations of the present invention is in the form of a pharmaceutically acceptable salt thereof. Such salt forms include for example and without limitation the sodium salt, potassium salt, calcium salt, magnesium salt and the like; as well as the hydrochloride, hydrobromide, sulfate, phosphate, formate, acetate, trifluoroacetate, maleate, tartrate, methanesulfonate, benzenesulfonate, p-toluenesulfonatemethanesulfonate salt forms, and the like. Preferably the active ingredient for use in accordance with the present invention is tranexamic acid.

An “immediate release oral dosage form” for purposes of the present invention is a dosage form which releases all of active ingredient (e.g., tranexamic acid) included therein within about 45 minutes when measured in vitro utilizing the USP 27 Dissolution Apparatus Type II Paddle Method @ 50 RPM in 900 ml water at 37±0.5° C.

The term “three times a day (TID) basis” for purposes of the present invention, means that the dosage regimen is to be administered three times a day, preferably on a schedule of every 8 hours.

The term “environmental fluid” is meant for purposes of the present invention to be an in-vitro dissolution fluid or gastrointestinal fluid.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

“Therapy” for excessive menstrual bleeding is defined for the purpose of this invention as one or more courses of treatment with an antifibrinolytic agent such as, but not limited to, tranexamic acid, aminocaproic acid, and any pharmaceutically acceptable salts, esters, derivatives, pro-drugs, metabolites, and analogues of any of the foregoing antifibrinolytic agents.

The term “heavy menstrual bleeding” is defined for purposes of the present invention as a perceived blood loss of at least heavy to very heavy which may correspond to a periodic blood loss of at least about 30 ml per cycle to as much as 1000 ml per cycle as measured by the alkaline hematin test. The periodic blood loss perceived or as measured with the alkaline hematin test may vary depending on the severity of the condition and the physiological make up of the individual patient. Therefore, heavy menstrual bleeding may include periodic blood losses of at least about 30 ml per cycle. Losses from between about 30 ml, about 40 ml, about 50 ml, about 60 ml, about 70 ml, about 80 ml, about 90 ml to about 300 ml are contemplated as are losses greater than 300 ml, such as for example, losses between about 300 ml to about 1000 ml.

### BRIEF DESCRIPTION OF THE DRAWINGS

FIG. 1 depicts mean plasma concentration-time profiles on a semi-log scale over 36 hours for the formulations of Examples 6 and 7 compared to IV tranexamic acid (Cyklokapron).

FIG. 2 depicts mean plasma concentration-time profiles on a linear scale over 36 hours for the formulations of Examples 6 and 7 compared to IV tranexamic acid (Cyklokapron).

FIG. 3 depicts the dissolution profiles of the modified release tranexamic acid formulation of Example 6; the immediate release tranexamic acid formulation of Example 7; the delayed release tranexamic acid formulation of Example 9, and the commercial Cyklokapron immediate release formulation of Example 16.

FIG. 4 is a listing of the Menorrhagia Impact Measures of the present invention.

FIG. 5 is a graph of Menorrhagia Instrument measure #1 percentage of patients and normals indicating each response at baseline (BL) and at one (1) month (M1) from Example 17.

FIG. 6 is a graph of the limitations of social and leisure activities (LSLA) in women with Heavy Menstrual Bleeding (HMB) in accordance with the treatment regimens administered in Examples 17A and 17B.

FIG. 7 is a graph of the mean menstrual blood loss change from the clinical studies of Examples 17A and 17B.

### DETAILED DESCRIPTION

The tranexamic acid (API) utilized in the formulations of the present invention is available from various manufacturers. The tranexamic acid particles utilized in the present invention may range from about 0.1 to about 550 microns. For example, the tranexamic acid particles may have a particle size range from < about 0.5 to about 520 microns.

The tranexamic acid particles utilized in the present invention may have a D₂₅ particle size distribution ranging from about 5 to about 15 microns, a D₅₀ particle size distribution ranging from about 14 to about 73 microns, and a D₇₅ particle size distribution ranging from about 30 to about 205 microns.

The particle size of the tranexamic acid utilized may also have a particle size range wherein about 1% of the particles are of a size greater than about 250 microns, about 8% of the particles are of a size of about 180 microns, about 9% of the particles are of a size of about 150 microns, about 4% of the particles are of a size of about 125 microns, about 20% of the particles are of a size of about 75 microns, about 14% of the particles are of a particle size of about 45 microns, and about 44% of the particles are of a particle size less than about 45 microns.

The tranexamic acid utilized may also have a particle size range wherein about 5% of the particles are of a size greater than about 250 microns, about 12% of the particles are of a size of about 180 microns, about 14% of the particles are of a size of about 150 microns, about 14% of the particles are of a size of about 125 microns, about 29% of the particles are of a size of about 75 microns, about 12% of the particles are of a particle size of about 45 microns, and about 14% of the particles are of a particle size less than about 45 microns.

The tranexamic acid utilized may also have a particle size range wherein about 2% of the particles are of a size greater than about 250 microns, about 7% of the particles are of a size of about 180 microns, about 9% of the particles are of a size of about 150 microns, about 4% of the particles are of a size of about 125 microns, about 20.5% of the particles are of a size of about 75 microns, about 16% of the particles are of a particle size of about 45 microns, and about 41.5% of the particles are of a particle size less than about 45 microns.

The tranexamic acid utilized may also have a particle size range wherein about 0% of the particles are of a size greater than about 250 microns, about 5% of the particles are of a size of about 180 microns, about 12% of the particles are of a size of about 150 microns, about 11% of the particles are of a size of about 125 microns, about 31% of the particles are of a size of about 75 microns, about 17% of the particles are of a particle size of about 45 microns, and about 24% of the particles are of a particle size less than about 45 microns.

The tranexamic acid utilized may also have a particle size range wherein about 20% of the particles are of a size of about 125 microns, about 20% of the particles are of a size of about 75 microns, about 20% of the particles are of a particle size of about 45 microns, and about 45% of the particles are of a particle size less than about 45 microns.

The dosage regimen typically listed for tranexamic acid in HMB (Heavy Menstrual Bleeding) therapy is 1-1.5 g per dose administered three-four times a day at the onset of copious menstrual bleeding and continued for the first 3-5 days of the menstrual cycle. However, the most frequently reported dosage regimen of tranexamic acid is 1 g four times a day (4 g per day) for HMB therapy outside of the US. Knowledge of this common regimen is supported by a careful review of the randomized controlled trials published in the medical literature, product labeling from other countries' regulatory authorities having the product approved for HMB therapy, utilization data from Sweden (Rybo 1991), correspondence and interviews with non-US clinicians having experienced with the product, and this regimen is currently the dosage being studied by the US Center for Disease Control (CDC) in women with HMB associated with bleeding disorders.

In immediate release formulations the entire dose and the soluble components in the dosage form dissolve in gastrointestinal fluid and present a high concentration of solutes for absorption. The most frequently reported adverse effects are primarily confined to the proximal gastrointestinal tract (nausea and vomiting). These adverse symptoms appear to be related to the drug load presented to the gastric mucosa, since this effect can be minimized by reducing the immediate-release oral formulation dose or administering the product slowly by the intravenous route. In certain embodiments, a lower incidence of proximal gastrointestinal adverse effects is obtained with the preferred oral modified release formulation or delayed release formulation (e.g., dosed 1.3 g every 8 hours) of the invention, e.g., because of the modified or delayed release properties of the drug product formulation.

The modified release oral formulations of tranexamic acid of the present invention provides a release of the drug which is slower than that of the immediate release 500 mg Cyklokapron product current marketed in Canada which provided a mean release rate of 100% by weight tranexamic acid released by about 15 minutes when measured utilizing USP 27 Dissolution Apparatus Type II paddle method @ 50 RPM in 900 ml water at 37±0.5° C.

In accordance with the present invention a modified release tranexamic acid tablet for oral administration is disclosed. Preferably, the tablet contains at least one material (defined herein as any substance other than the active, i.e., tranexamic acid) which minimizes or eliminates the adverse gastrointestinal side effects in patients, for example, women dosed with oral tranexamic acid for treatment of menorrhagia.

A modified release product is defined by the United States Pharmacopeia (USP) as including delayed release products and extended-(controlled) release products. One embodiment is an extended release formulation, also called a sustained release formulation or a controlled release formulation. Extended, controlled, or sustained release formulations decrease the concentration of tranexamic acid and excipients dissolved in the stomach fluids after dosing by controllably releasing tranexamic acid over a period of time, as opposed to immediate release formulations which release the entire dose of tranexamic acid all at once. In immediate release formulations the entire dose and the soluble components in the dosage form dissolve in gastric fluid and present a high concentration of solutes for absorption.

Another embodiment is a delayed release formulation. The definition of a delayed release dosage form used herein is that from the USP, Chapter 1151 Pharmaceutical Dosage Forms—Tablets. In certain embodiments, where the delayed release formulation is a tablet, the tablet contains one or more coatings, intended to delay the release of tranexamic acid until the tablet has passed through the stomach (enteric coatings). A delayed release tablet is a dosage form that releases tranexamic acid at a time later than immediately after administration, that is, it exhibits a lag time in quantifiable plasma tranexamic concentrations. Preferably, one or more coating(s) delays the release of tranexamic acid until the dosage form has passed through the acidic medium of the stomach.

Delayed release formulations minimize or prevent release of tranexamic acid in the stomach and delay its release until the dosage form has emptied from the stomach into the small intestine. Delayed release formulations include enteric-coated tablets, enteric-coated capsules, enteric-coated granules, enteric-coated beads, and enteric-coated spheres (commonly referred to as “tiny little time pills” or multiparticulate dosage forms).

The enteric coating is stable under the acidic conditions in the stomach and releases tranexamic acid only in the less acidic or substantially neutral medium of the intestine, (e.g., at pH about 5.5 to about 7.5). It disintegrates, erodes, or dissolves, releasing tranexamic acid only when it encounters the higher pH of the intestine. Enteric-coated formulations substantially prevent dissolution of tranexamic acid in the relatively lower pH of the stomach. Both extended release and delayed release formulations are modified-release forms that thus minimize or prevent gastrointestinal reactions and side effects that occur when a dose of tranexamic acid reaches the stomach and unimpededly begins to dissolve.

As used herein, the terms extended release formulations, controlled release formulations, or sustained release formulations are used to describe drug product formulations designed to release tranexamic acid over a prolonged period of time. The definition of an extended release tablet used herein is that from the USP, Chapter 1151, as previously cited. The tablet is formulated in such a manner as to make tranexamic acid available over an extended period of time following ingestion. Expressions such as “prolonged action”, “repeat-action”, and “sustained release” also describe such a dosage form. Extended release dosage forms typically allow reduced dosing frequency as compared to when tranexamic acid is present in an immediate release dosage form. These extended release dosage forms may also reduce fluctuations in plasma tranexamic acid concentrations. Extended release dosage forms may be prepared as a tablet, capsule, granule, pellet or suspension, and may be packaged into capsules, sachets, etc. They may be prepared by any formulation technique where release of the active substance (tranexamic acid) from the dosage form is modified to occur at a slower rate than that from an immediate release product. In these formulations, tranexamic acid release occurs both in the stomach and intestine, but at a slower rate so that a bolus of dissolved drug does not reach the lining of the stomach or intestine and cause adverse effects, or adverse effects occur with a lower intensity or frequency because of the lower concentration of tranexamic acid. Hence, adverse effects are reduced, minimized or eliminated.

The modified release dosage forms of the present invention may be prepared as; tablets, capsules, granules, pellets, powders, dragees, troches, non-pariels, pills or encapsulated suspension, and may be packaged into capsules, sachets, etc. Such dosage forms may be prepared by any formulation technique where release of the active substance (tranexamic acid) from the dosage form is modified to occur at a slower rate than from an immediate release product. In these formulations, tranexamic acid release occurs in the stomach and/or intestine, but at a slower rate so that a bolus of dissolved drug does not reach the lining of the stomach and cause adverse effects, or adverse effects occur with a lower intensity or frequency because of the lower concentration of tranexamic acid. Hence, adverse effects are preferably reduced, minimized or eliminated.

Methods of preparing modified release formulations are known to one skilled in the art and are found in Modified Release Drug Delivery Technology, Rathbone, Hadgraft, and Roberts, Eds., Drugs and the Pharmaceutical Sciences, Vol. 126, Marcel Dekker Inc, New York, 2003; Modern Pharmaceutics, Third Edition, Banker and Rhodes, Eds., Drugs and the Pharmaceutical Sciences, Vol. 72, Marcel Dekker Inc., New York, 1996; Sustained and Controlled Release Drug Delivery Systems, Robinson, Ed., Drugs and the Pharmaceutical Sciences, Vol. 6, Marcel Dekker Inc., NY 1978; Sustained Release Medications, Chemical Technology Review No. 177, Johnson, Ed., Noyes Data Corporation 1980; Controlled Drug Delivery, Fundamentals and Applications, Second Edition, Robinson and Lee, Eds., Marcel Dekker Inc., New York, 1987, and as described in U.S. Pat. No. 6,548,084, which is expressly incorporated by reference herein in its entirety. The terms extended release formulation, controlled release formulation, and sustained release formulation are used interchangeably herein, unless indicated otherwise.

An extended release form, one example of a modified release form, makes tranexamic acid available over an extended period of time after ingestion. Extended release dosage forms coupled with the digestion process and the absorption process in the gastrointestinal tract cause a reduction in the amount of tranexamic acid in solution in the gastrointestinal tract compared to dosing tranexamic acid presented as a conventional dosage form (e.g., as a solution, or as an immediate release dosage form). The extended release formulation may be verified by in vitro dissolution testing and in vivo bioequivalence documentation, according to Food and Drug Administration standards, e.g, as set forth at www.fda.gov, 21 CFR §314, 320, and also according to the USP. Briefly, in vitro dissolution is conducted on twelve individual dosage units. Multipoint dissolution profiles are obtained using discriminating combinations of apparatus, agitation speed, and medium. A surfactant may be used if justified. Sampling times are selected to define the release characteristics of the dosage form and to assure batch to batch reproducibility. Suitable equipment for dissolution testing is specified in USP 23 Apparatus 1 (rotating basket); Apparatus 2 (rotating paddle); Apparatus 3 (reciprocating cylinder*), Apparatus 4 (flow-through cell*); and Apparatus 5 (reciprocating disk*) (*modified testing conditions are used). Rotation speeds of 50 rpm, 100 rpm and 150 rpm are used with baskets, and 50 rpm, 75 rpm and 100 rpm are used with paddles. The temperature is 37° C.±0.5° C. The dissolution volume is 500 ml to 1000 ml. The dissolution medium is aqueous, at various pH values. The sampling schedule is such that adequate sampling is performed until either 80% of tranexamic acid is released or an asymptote is reached.

Tranexamic acid modified release tablets may be formulated to provide a dose of tranexamic acid, typically about 500 mg to about 2 grams from one to two tablets, within about the first one to two hours after the tablet is ingested. Thus, tranexamic acid release occurs at a designed rate over a period e.g., about 60 minutes to about 120 minutes. The rate of tranexamic acid release over this period of time is designed to provide a reduced concentration of tranexamic acid in the stomach while allowing the absorption of tranexamic acid to occur throughout the gastrointestinal tract. Absorption of tranexamic acid typically begins as soon as tranexamic acid is released from the dosage form and is dissolved in the gastrointestinal fluids contacting the membranes which line the gastrointestinal tract. The rate of release of tranexamic acid from the dosage form, the secretion of gastrointestinal fluid, and the absorption of drug by the gastrointestinal mucosa help to maintain low concentrations of drug in the gastrointestinal fluids. The lowered concentrations preferably result in lower intensity, frequency, and/or severity of gastrointestinal adverse side effects. The designed rate of release of tranexamic acid from the dosage form in the stomach and the upper small intestine, the natural emptying of gastric juice containing any dissolved tranexamic acid from the stomach, and the absorption of tranexamic acid from a larger segment of the gastrointestinal tract (i.e., both the stomach and the small intestine, rather than the stomach only or the lower portion of the small intestine if any modified release dosage form with a longer release time was used), preferably results in reduced levels of dissolved tranexamic acid in the region of the gastrointestinal tract proximal or distal to the dosage form. Reduced concentrations of tranexamic acid along the gastrointestinal tract preferably provide a reduction in adverse gastrointestinal effects associated with oral tranexamic acid therapy.

As used herein, alleviation of adverse effects using these formulations indicates any relief in one or more symptoms, such as decrease in incidence, severity, or duration of symptoms, and is not limited to absence of symptoms or elimination of symptoms. Thus, treatment includes any decrease in incidence, duration, intensity, frequency, etc. of adverse gastrointestinal symptoms including, but not limited to, headache, nausea, vomiting, diarrhea, constipation, cramping, bloating, and combinations thereof. The formulations may reduce symptoms at any time during tranexamic acid therapy, but minimized adverse effects are particularly noted immediately or shortly after dosing, that is, within the first few hours after dosing. As used herein, adverse gastrointestinal effects and side effects are used interchangeably to indicate nontherapeutic effects (i.e., not relating to any possible beneficial effects due to tranexamic acid), ranging from unpleasant but tolerable sensations to severe gastrointestinal symptoms. As used herein, the terms oral formulations, ingestable formulations, and orally administered formulations are used interchangeably and include any dosage forms which are ingested by mouth, including, but not limited to, tablets, pills, liquids, gelcaps, softgels, dragees, capsules, powders, granules, pellets, etc.

Modified release formulations of tranexamic acid include tablets, pellets, granules, capsules, or other oral dosage forms prepared in such a way to release tranexamic acid in a designed manner. In certain embodiments, the modified release material is a gel-forming polymer, a hydratable polymer, a water soluble polymer, a water swellable polymer, or mixtures thereof.

As used herein, the term delayed release formulation indicates any formulation technique where release of the active substance (tranexamic acid) from the dosage form is modified so that release occurs at a later time than that from a conventional immediate release product. One example of a delayed release formulation is an enteric coated formulation. Enteric coatings on the dosage form are intended to control the region of the gastrointestinal tract where dissolution and subsequent absorption of tranexamic acid from the enteric coated dosage form occurs. Enteric coatings can be prepared to substantially prevent dissolution of the dosage form contents in the stomach. These coatings function by incorporating materials in the enteric coating which allow the enteric coating to remain substantially intact in the acidic environment of the stomach. This substantially intact enteric coating minimizes or prevents the dissolution of tranexamic acid in stomach contents. Enteric coatings are formulated to release the contents of the dosage form when the pH of the gastrointestinal fluid increases. This increase in pH typically occurs when the dosage form passes out of the stomach into the small intestine. That is, the coating remains intact in the relatively more acidic stomach pH (pH≦3) and disintegrates, dissolves, or is otherwise removed in the relatively less acidic pH of the intestine (pH of from about 3 through about 5 to about 7 for the upper regions of the small intestine and pH values from about 7 to about 8.5 in the lower regions of the intestines). Formulations can be prepared using enteric coatings intended to release tranexamic acid at pH values of about 5.5 to about 6.5 or at higher pH values that typically occur in the lower regions of the intestines. In those delayed release formulations intended to dissolve at pH 5.5 to about 6.5 or higher, tranexamic acid release occurs substantially only upon reaching the duodenum (the upper portion of the small intestine) so that substantially no tranexamic acid is released in the stomach, thus minimizing or eliminating adverse effects.

Tranexamic acid formulated as delayed release tablets may contain an enteric coating which disintegrates, dissolves, or erodes at neutral or slightly acidic or slightly alkaline pH, and thereby allows dissolution of tranexamic acid upon leaving the stomach, that is, upon stomach emptying into the small intestine. The release of tranexamic acid in the intestine reduces gastrointestinal side effects associated with the large dose of tranexamic acid quickly released into the stomach. Patients treated with enteric coated formulations of tranexamic acid for delayed release should be cautioned to not consume antacids while under tranexamic therapy, because antacids will change the stomach pH and thus alter the site of tablet dissolution or disintegration. Other types of delayed release formulations are available, and the above example is not limiting.

A delayed release form, another example of a modified release form, makes tranexamic acid available at a time other than immediately following oral administration. As for extended release formulations, delayed release formulations may be verified by in vitro dissolution testing and in vivo bioequivalence documentation according to the standard available as previously set forth (USP). When the guidance refers to dissolution testing in addition to application/compendial release requirements, the dissolution test should be performed in 0.1 N HCl for two hours (acid stage), followed by testing in USP buffer media at a pH range between 4.5 to 7.5 (buffer stage) under standard (application/compendial) test conditions and increased agitation speeds using the application/compendial test apparatus. For the rotating basket method (Apparatus 1) a rotation speed of 50 rpm, 100 rpm, and 150 rpm may be used, and for the rotating paddle method (Apparatus 2) a rotation speed of 50 rpm, 75 rpm, and 100 rpm may be used. Multipoint dissolution profiles may be obtained during the buffer stage of testing. Adequate sampling should be performed, e.g., at 15 min, 30 min, 45 min, 60 min, 120 min (following the time from which the dosage form is placed in the buffer), until either 80% of the drug is released or an asymptote is reached.

Methods of preparing delayed release formulations are known to one skilled in the art and are found in, for example, Remington's Pharmaceutical Sciences 16^{th} Edition, Mack Publishing Company 1980, and the references cited for extended release formulations.

Delayed release formulations may be enteric coated tranexamic acid tablets or enteric coated granules. These tablets may be prepared by coating compressed tablets with a delayed release material such as a commercial or specially formulated enteric film coat, for example, a wax, a polymer, and/or other additives such as colorants and pigments that form a pH-sensitive matrix that meets (USP) and Food and Drug Administration (FDA) requirements for enteric coated tablets. The enteric coating permits disintegration of the tranexamic acid tablets and dissolution of tranexamic acid as a result of the pH change between the stomach and the duodenum. Tablet excipients, such as delayed release materials, which inhibit rapid release of tranexamic acid in the stomach and which promote dissolution and release in the intestine may also be used. These include, but are not limited to, phthalic acid derivatives such as phthalic acid derivatives of vinyl polymers and copolymers, hydroxyalkylcelluloses, alkylcelluloses, cellulose acetates, hydroxyalkylcellulose acetates, cellulose ethers, alkylcellulose acetates and partial esters thereof, and polymers and copolymers of lower alkyl acrylic acids and lower alkyl acrylates and partial esters thereof. Commercial preparations intended for the enteric coating of tablets, capsules, and granules are available from Degussa (Parsippany, N.J.) and Colorcon (West Point, Pa.). In one embodiment, the polymers are methacrylic acid copolymers. These are copolymers of methacrylic acid with neutral acrylate or methacrylate esters such as ethyl acrylate or methyl methacrylate, for example, methacrylic acid copolymer, Type C, USP (a copolymer of methacrylic acid and ethyl acrylate having between 46.0% and 50.6% methacrylic acid units), commercially available from Rohm Pharma as Eudragit® L 100-55 (as a powder) or L30D-55 (as a 30% dispersion in water). In another embodiment, the polymers are hydroxypropyl cellulose phthalate, hydroxypropyl methylcellulose phthalate, cellulose acetate phthalate, polyvinylacetate phthalate, polyvinylpyrrolidone phthalate, and the like. One or more pH-dependent excipient(s) are present in amounts ranging from about 1% by weight to about 20% by weight, from about 5% by weight to about 12% by weight, or in an amount of about 10% by weight.

The quantity of pH dependent excipients is sufficient to produce a delayed release formulation from which the release rate of tranexamic acid is controlled such that at a pH below about 5 the rate of dissolution is significantly retarded. For methacrylic acid copolymer, type C, USP (Eudragit® L 100-55), a quantity of pH dependent polymer coating may be applied to tablets in the range between about 2% to about 15% by weight (dry basis). In another embodiment, the range is between about 3% to about 6% by weight (dry basis). The pH dependent polymer may have from about 1% to about 20% of the methacrylic acid carboxyl groups neutralized. In one embodiment about 3% to about 6% of the binder methacrylic acid carboxyl groups are neutralized. One or more pH independent excipients may be present in amounts ranging from about 1% by weight to about 10% by weight, from about 1% by weight to about 3% by weight, or in an amount of about 2% by weight. Film-forming or viscosity enhancing agents may also be present, such as hydroxypropyl methylcellulose, hydroxypropyl cellulose, methylcellulose, polyvinylpyrrolidone, neutral poly(meth)acrylate esters, and the like.

Excipients may be admixed so as to form a homogeneous mixture with tranexamic acid and the pH dependent binder. Excipients include pH independent binders or film-forming agents such as hydroxypropyl methylcellulose, hydroxypropyl cellulose, methylcellulose, polyvinylpyrrolidone, neutral poly(meth)acrylate esters (e.g., the methyl methacrylate/ethyl acrylate copolymers sold as Eudragit® (Rohm Pharma), starches, gelatin, sugars such as glucose, sucrose, and mannitol, silicic acid, carboxymethylcellulose, and the like, diluents such as lactose, mannitol, dry starch, microcrystalline cellulose and the like, surface active agents such as polyoxyethylene sorbitan esters, sorbitan ethers, and the like, coloring agents, flavoring agents, lubricants such as talc, calcium stearate, and magnesium stearate, solid polyethylene glycols, sodium lauryl sulfate, and other tableting aids. These excipients may be combined with tranexamic acid to form delayed release tablets.

In certain embodiments of the formulation includes tranexamic acid in the range of about 50% by weight to about 95% or more by weight of the formulation. In other embodiments, tranexamic acid is in the range of about 60% by weight to about 90% by weight, or about 60% by weight to about 80% by weight of the formulation. The remaining weigh may be made up of the modified release material and additional excipients.

In certain embodiments, the pH dependent binder may be in the range of about 5% by weight to about 40% by weight, about 5% by weight to about 25% by weight, or about 5% by weight to about 15% by weight. The remaining weight may be made up of tranexamic acid, pH independent binders, fillers, or other excipients.

To prepare delayed release tablet formulations, the agent to control or delay the release of tranexamic acid may be incorporated into the tablet matrix or coated onto the tablet surface or both. Tablet formulations prepared with the pH dependent excipient added as a binder in the tablet matrix are formulated by granulating a blend of powders composed with the pH dependent binder. Alternatively, the pH dependent binder may be added as a powder and wet granulated by addition of a solvent to the powder blend. The powder blend is formed by combining portions of the powdered components that make up the tablet. These powders are intimately mixed by dry-blending. The dry blended mixture is granulated by wet mixing of a solution of a binding agent with the powder blend. The time for such wet mixing may be controlled to influence the dissolution rate of the formulation. For example, the total powder mix time, that is, the time during which the powder is granulated, may range from about 1 min to about 10 min, or from about 2 min to about 5 min. Following granulation, the particles are removed from the granulator and placed in a fluid bed dryer, a vacuum dryer, a microwave dryer, or a tray dryer for drying. Drying conditions are sufficient to remove unwanted granulating solvent, typically water, or to reduce the amount of granulating solvent to an acceptable level. Drying conditions in a fluid bed dryer or tray dryer are typically about 60° C. The granulate is dried, screened, mixed with additional excipients such as disintegrating agents, flow agents, or compression aids and lubricants such as talc, stearic acid, or magnesium stearate, and compressed into tablets.

The tablet that contains a delayed release agent within the tablet matrix may be coated with an optional film-forming agent. This applied film may aid in identification, mask an unpleasant taste, allow desired colors and surface appearance, provide enhanced elegance, aid in swallowing, aid in enteric coating, etc. The amount of film-forming agent may be in the range of about 2% tablet weight to about 4% tablet weight. Suitable film-forming agents are known to one skilled in the art and include hydroxypropyl cellulose, cellulose ester, cellulose ether, one or more acrylic polymer(s), hydroxypropyl methylcellulose, cationic methacrylate copolymers (diethylaminoethyl) methacrylate/methyl-butyl-met-hacrylate copolymers such as Eudragit E® (Rohm Pharma) and the like. The film-forming agents may optionally contain colorants, plasticizers, fillers, etc. including, but not limited to, propylene glycol, sorbitan monooleate, sorbic acid, titanium dioxide, and one or more pharmaceutically acceptable dye(s).

In certain embodiments, the tranexamic acid tablets of the invention are coated with a modified release material. In certain embodiments, tranexamic acid tablets are formulated by dry blending, rotary compacting, or wet granulating powders composed of tranexamic acid and tablet excipients. These powders are compressed into an immediate release tablet. Coating this immediate release tablet with a modified release material as described herein renders this tranexamic acid tablet as a modified release tablet.

In addition to the modified release material and/or delayed release material, the formulations of the invention may also contain suitable quantities of other materials, e.g. preservatives, diluents (e.g., microcrystalline cellulose), lubricants (e.g., stearic acid, magnesium stearate, and the like), binders (e.g., povidone, starch, and the like), disintegrants (e.g, croscarmellose sodium, corn starch, and the like), glidants (e.g., talc, colloidal silicon dioxide, and the like), granulating aids, colorants, and flavorants that are conventional in the pharmaceutical art. Specific examples of pharmaceutically acceptable excipients that may be used to formulate oral dosage forms are described in the Handbook of Pharmaceutical Excipients, American Pharmaceutical Association (2003), incorporated by reference herein.

In one embodiment, tranexamic acid tablets are coated with an enteric film coat. Tranexamic acid tablets are formulated by dry blending, rotary compacting, or wet granulating powders composed of tranexamic acid and tablet excipients. These powders are compressed into an immediate release tablet. Coating this immediate release tablet with an enteric coating renders this tranexamic acid tablet as a delayed release tablet.

Extended release formulations of tranexamic acid include tablets, pellets, granules, capsules, or other oral dosage forms prepared in such a way to release tranexamic acid in a controlled manner.

Extended release tranexamic acid tablets are prepared by adding a gel-forming or hydratable polymer to a tranexamic tablet composition. Suitable gel-forming or hydratable polymers include, but are not limited to, hydroxypropylcellulose, hydroxypropylmethylcellulose, carboxymethylcellulose, polyvinyl alcohol, etc. This provides a compressed tablet that may or may not be film-coated. The tablet releases tranexamic acid by diffusion of tranexamic acid through the tablet matrix, or by erosion of the tablet matrix, or by a combination of diffusion from and erosion of the tablet matrix. Alternatively, water-swellable polymers may be used to form the tablet matrix. Tablets formed with water swellable polymers release tranexamic acid by diffusion of tranexamic acid through the tablet matrix, or by erosion of the tablet matrix, or by a combination of diffusion from and erosion of the tablet matrix. One or more water-soluble hydrophilic polymer(s) may also be used. These include polyvinylpyrrolidine, hydroxypropyl cellulose, hydroxypropylmethylcellulose, now referred to as hypromellose (e.g., Methocel™, Dow Chemical Company), methyl cellulose, vinyl acetate/crotonic acid copolymers, methacrylic acid copolymers, maleic anhydride/methyl vinyl ether copolymers, derivatives thereof and mixtures thereof. In various embodiments, the polymer is hydroxypropyl cellulose or hydroxypropylmethylcellulose. The polymer may be hydroxypropyl-methyl cellulose with a viscosity ranging from about 50 cps to about 200 cps. The polymer may be hydroxypropylmethylcellulose with a viscosity of 100 cps, commercially available as Methocel™ K 100 LV (Dow Chemical Company). The amount of polymer in the composition may be in the range of about 5% by weight to about 50% by weight of the composition. In various embodiments, the polymer is in the range of about 10% by weight to about 35% by weight of the composition, or about 10% by weight to about 30% by weight of the composition.

The tablet matrix may also contain soluble and insoluble components to aid in the formulation and/or the extended release rate of tranexamic acid. The release process may be adjusted by varying the type, amount, and the ratio of the tablet ingredients to produce the desired dissolution profile, as known to one skilled in the art. A coating may be a partially neutralized pH-dependent binder that controls the rate of tranexamic acid dissolution in aqueous media across the range of pH in the stomach, which has a pH of about 2, and the intestine, which has a pH of about 5.5. One or more pH dependent binders are used to control the dissolution profile so that tranexamic acid is released slowly and continuously as the formulation passes through the stomach and gastrointestinal tract.

In one embodiment, compressed extended release tablets are formulated to comply with USP criteria and to be of such a size and shape to be easy to swallow. The size of the tablet will depend upon the dose of tranexamic acid that is needed to provide adequate therapy and the particular formulation and excipients that are selected to provide the physical properties necessary for tableting and for extended release. In various embodiments, a compressed extended release tablet contains from about 500 mg to about 1 gram of tranexamic acid, or from about 600 mg to about 750 mg of tranexamic acid. The daily dose of tranexamic acid may be achieved by taking one or two tablets at each dosing time.

In certain embodiments, the tranexamic acid included in the dosage form is from about 375 mg to about 1500 mg, preferably from about 375 mg to about 1000 mg. In one embodiment, the dose of tranexamic acid per tablet is in the range of about 500 mg to about 1000 mg for tablets and from about 500 mg to about 1500 mg for a sachet filled with granules. In another embodiment, the dose of tranexamic acid is in the range of about 3 grams/day to about 6 grams/day in three or four divided doses. As an example, a total daily dose of 3 grams tranexamic acid may be divided into three doses of one tablet each with each tablet containing 1 gram tranexamic acid, or may be divided into four doses of one tablet each with each tablet containing 0.75 gram tranexamic acid. As another example, a total daily dose of 4 gram tranexamic acid may be divided into three doses of two tablets at each dose with each tablet containing 0.666 gram tranexamic acid, or may be divided into four doses of one tablet each with each tablet containing 1 gram tranexamic acid. As another example, a total daily dose of 5 gram tranexamic acid may be divided into three doses of one tablet each with each tablet containing 1.66 gram tranexamic acid, or may be divided into four doses of two tablets each with each tablet containing 0.625 gram tranexamic acid. As another example, a total daily dose of 6 gram tranexamic acid may be divided into three doses of two tablets each with each tablet containing 1 gram tranexamic acid, or may be divided into four doses of two tablets each with each tablet containing 0.75 gram tranexamic acid. For ease of swallowing, the dose of tranexamic acid taken at each dosing time may be delivered by taking multiple tablets. For example, the 4 gram daily dose may be delivered by taking two 666.67 mg tablets three times a day or, two 500 mg tablets four times a day. Similarly, the 3 gram daily dose may be achieved by taking two 550 mg tablets three times a day or two 375 mg tablets four times a day. Alternatively, for ease of reference, a dose of 600 mg, 650 mg, or 700 mg of tranexamic acid per tablet may be used. In a preferred embodiment, a total daily dose of 3900 mg/day is administered in three divided doses of 1300 mg of two tablets at each dose with each tablet containing 650 mg of tranexamic acid. Alternatively, each dose may be delivered by taking granules containing the prescribed amount of tranexamic acid presented in a convenient unit dose package. Such examples are not limiting and other doses within these ranges will be appreciated by those skilled in the art.

Since tranexamic acid is primarily eliminated via the kidneys by glomerular filtration with more than 95% excreted unchanged drug in the urine, dosage adjustment may be recommended. The table below lists some recommended dosage adjustments for renal impairment:

| Serum | Estimated | | | |
|---|---|---|---|---|
| Creatine | GFR* | | | |
| (mg/dl) | (ml/min) | Adjusted dose | Total daily dose | |
| 1.4 to 2.8 | 30-60 | 1.3 g (two 650 mg tablets) | 2.6 | g |
| | | BID | | |
| 2.8 to 5.7 | 15-30 | 1.3 g (two 650 mg tablets) | 1.3 | g |
| | | QD | | |
| >5.7 | <15 | 1.3 g (two 650 mg tablets) | 0.65 | g |
| | | every 48 hours of 650 mg | | |
| | | (one tablet) every 24 hours | | |

Alternatively, extended release or delayed release tranexamic acid formulations may be administered as a multiparticulate formulation (e.g., by pellets or granules in a sachet). Extended release tranexamic acid pellets or granules may be prepared by using excipients to control the release of tranexamic acid from the granule or pellet matrix. Extended release preparations may also be formulated using coatings to control the release of tranexamic acid from the granule or pellet. Delayed release formulations may be prepared by incorporating excipients to control the release of tranexamic acid in the matrix of the granule or pellet, or as coating materials on the surface of the granule or pellet. U.S. Pat. No. 6,433,215, which is expressly incorporated by reference herein in its entirety, discloses a method of building layers of drug and binder on sugar spheres and coating them with a membrane to form a film coating. Such a coating may be used for either an extended release formulation or a delayed release formulation, and/or for pharmaceutical elegance. U.S. Pat. Nos. 5,650,174; 5,229,135; and 5,242,337, each of which is expressly incorporated by reference herein in its entirety, disclose variations on fabricating a pellet or nonpareil dosage form. Spheres are filled into packets, termed sachets, which are filled by weight to contain the prescribed dose of drug. Multiparticulates may be coated with an extended release coating or a delayed release coating, as disclosed in U.S. Pat. No. 6,066,339, which is expressly incorporated by reference herein in its entirety. Coated multiparticulates may be packaged in capsules or sachets. The formulation of granules or pellets for extended or delayed release is described in Multiparticulate Oral Drug Delivery, Ghebre-Sellassie, Ed. in Drugs and the Pharmaceutical Sciences, Vol. 65, Marcel Dekker Inc., NY, 1994 and in the relevant parts of the references for extended release formulations and delayed release formulations previously cited and the relevant portions incorporated herein by reference. In certain embodiments the multiparticulates are incorporated into a capsule or are formed into a tablet (e.g., such as by compression with additional excipients).

The inventive tranexamic acid formulations may be used for additional indications other than menorrhagia.

Additional tranexamic acid formulations are disclosed in U.S. patent application Ser. Nos. 12/220,241, filed Jul. 23, 2008 and 12/228,489, filed Aug. 13, 2008, the disclosures of which are hereby incorporated by reference in their entirety.

### Menorrhagia Instrument

With regard to the treatment of menorrhagia (Heavy Menstrual Bleeding) studies of the safety and efficacy of the antifibrinolytic tranexamic acid were conducted. As part of these studies a diagnosis and treatment instrument (Menorrhagia Instrument; MI) was designed. The instrument reliably identifies and monitors heavy menstrual bleeding patients and can be used in conjunction with an antifibrinolytic agent to diagnose and monitor the treatment of heavy menstrual bleeding.

A Menorrhagia Instrument (MI) of the invention reliably captures the diagnosis and treatment of the disease by measuring the impact of treatment on the symptoms associated with heavy menstrual bleeding. The information obtained from individual patient responses to the measures described in the methods of the present invention correlates to blood loss as measured by the alkaline hematin test. For example, data from the measures of social, leisure and/or physical activity symptoms, correlate with the volume of blood loss, and the change in the intensity of these symptoms correlates with the change in volume of blood lost, thus providing a measurement for the successful diagnosis and evaluation of treatment of bleeding disorders.

The instrument of the present invention measures specific aspects of the patient's monthly menstrual period. The measures correlate with the diagnosis of heavy menstrual bleeding and with the course of antifibrinolytic treatment. Further each of the measures individually correlate with quantity of blood loss as measured by the alkaline Hematin test. The symptomatic measures include: 1) a functional assessment measure; and ii) a pharmacology (or therapy assessment) measure.

The functional assessment measure of symptoms is further factored into segments which include 1) a measure of functional impairment generally; 2) impairment of necessary activities; and 3) impairment of discretionary activities.

The pharmacology domain provides an assessment of the severity of the menstrual period.

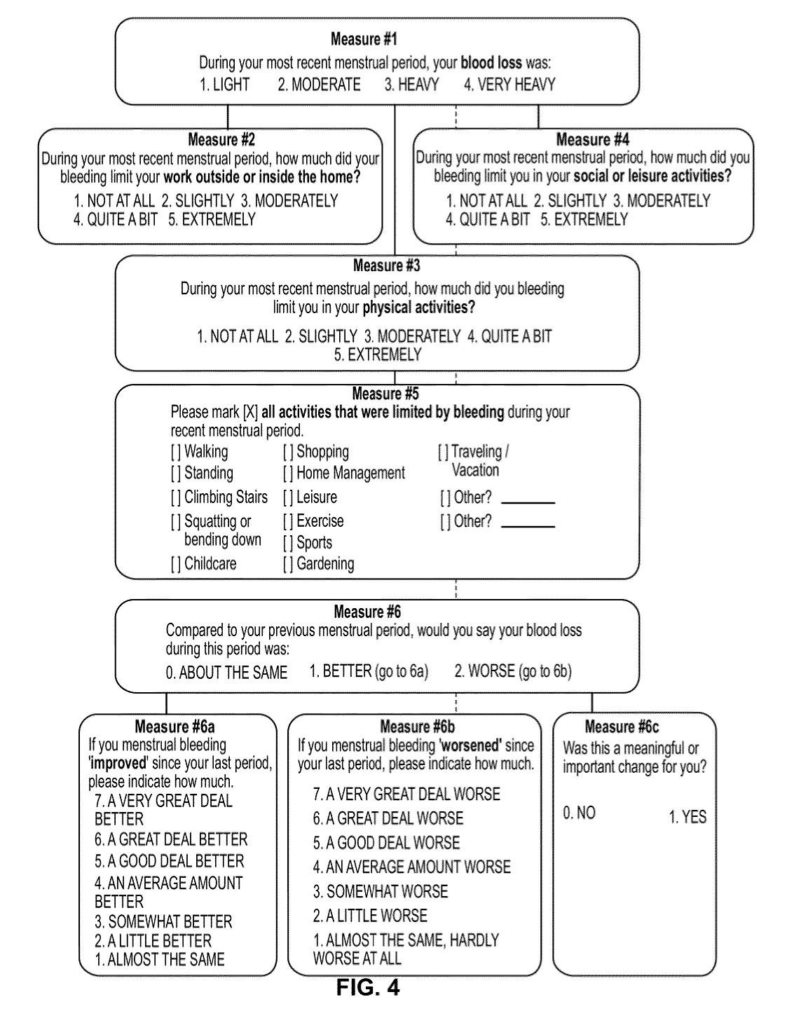

Specific symptomatic measures may be directed to an initial patient assessment and to the treatment period (pharmacology measure). Examples of specific measures would include examples of initial patient assessment measures (measures 1-4 listed in the Menorrhagia Instrument of FIG. 4); and therapy assessment measures (measures 1-4 together with measures 6, 6a, 6b and 6c contained in the Menorrhagia Instrument of FIG. 4).

In certain embodiments, the present invention is directed to a method of diagnosing and treating heavy menstrual bleeding, wherein the initial diagnoses of heavy menstrual bleeding is accomplished by evaluation of the most recent menstrual period on the basis of one, some or all of the prescribed symptomatic measures of FIG. 4. Measures which may be used as part of the initial patient assessment include, for example: a) determining a patient's perceived blood loss during their most recent menstrual period; b) determining how much the patient's blood loss limited their work outside and inside the home; c) determining how much the patient's blood loss limited their physical activities; d) determining how much the patient's blood loss limited their social and leisure activities; and e) determining the specific activities that were limited by the patient's blood loss.

The assessment of the patient's perceived blood loss during their most recent menstrual period may include an inquiry such as “during your most recent menstrual period, your blood loss was”. The assessment may then quantify the patient response as a blood loss that was: i) light, ii) moderate, iii) heavy, or iv) very heavy. Alternatively, the measure may be quantified in terms of a scale of from one to four where one represents light, two represents moderate, three represents heavy and four represents very heavy.

The assessment of a patient's limitation due to the blood loss may include and evaluation of the patient's blood loss limitation on physical activities and/or how much the patient's blood loss limited their social and leisure activities. Assessment of the limitations on work, physical, social and leisure activities may be quantitated as: i) not at all, ii) slightly, iii) moderately, iv) quite a bit, or v) extremely. Alternatively the measure may be quantified in terms of a scale of from one to five where one represents not at all, two represents slightly, three represents moderately, four represents quite a bit, and five represents extremely.

Activities limited may include, but are not limited to, walking, standing, climbing stairs, squatting or bending down, playing with children and attending school activities. Home management activities include, but are not limited to, cooking, cleaning, yard work, and laundry. Leisure activities may include, but are not limited to, dancing, dinner, and movies. Sports activities may include, but are not limited to, tennis, golf, running, swimming, hiking, biking, boating, baseball, softball, basketball, soccer, fencing, volleyball, and other sports related activities.

Once the initial patient assessment measures have been completed and the patient has been identified as in need of treatment, the patient is administered a therapeutically effective treatment regimen of an antifibrinolytic agent. Suitable antifibrinolytic agents contemplated for use in the present invention include, but are not limited to tranexamic acid, aminocaproic acid, pharmaceutically acceptable salts, esters, derivatives, pro-drugs, metabolites, and analogues of any of the foregoing antifibrinolytic agents.

In certain embodiments the preferred antifibrinolytic agent is tranexamic acid. The tranexamic acid utilized in the present invention can be formulated into any suitable dosage form. Preferably, the tranexamic acid is in the form of an immediate release tranexamic acid formulation which release is characterized by providing in vitro no more than 95% by weight of the tranexamic acid or a pharmaceutically acceptable salt thereof in about 15 minutes when measured according to USP 27.

When the preferred antifibrinolytic is tranexamic acid, the therapeutically effective treatment regimen contemplated by the present invention includes administration of a single dose of a tranexamic acid ranging from about 650 mg to about 1300 mg three (3) times a day for at least one day of menstruation, but not more than five days (or 15 single doses). The treatment regimen may be administered for at least one day; for at least the first two days, for at least the first three days, for days two through three, for days two to three, for the duration of menstruation.

In certain embodiments the tranexamic acid treatment regimen for treating the heavy menstrual bleeding includes administration of a single dose of about 650 mg to about 1.3 gm of a immediate release formulation of the invention three (3) times a day, wherein the immediate release formulation provides, in vitro, no more than 95% by weight of the tranexamic acid or a pharmaceutically acceptable salt thereof in about 15 minutes when measured according to USP 27.

In certain other embodiments, the present invention is directed to a method of evaluating the effectiveness of a treatment regimen administered for heavy menstrual bleeding and the amelioration of symptoms associated with heavy menstrual bleeding including limitations on social, leisure, and physical activities.

Evaluation of the effectiveness of the treatment regimen can be initiated at the end of the patient's menstrual period, but prior to completion of the menstrual cycle. The post-menstruation measures provide in part the pharmacology (or therapy assessment) measure described above.

The pharmacology assessment may begin with one or more of the same series of measures utilized during the initial patient assessment, which include: a) determining a patient's perceived blood loss volume during their most recent menstrual period; b) determining how much the patient's blood loss limited their work outside and inside the home; c) determining how much the patient's blood loss limited their physical activities; d) determining how much the patient's blood loss limited their social and leisure activities; e) determining the specific activities that were limited by the patient's blood loss.

Alternatively, an evaluation of the effectiveness of the treatment regimen may require determining the change in the patient's perceived blood loss during the most recent menstrual period in comparison to the blood loss during the patient's previous menstrual period, measure 1 of FIG. 4 and/or an assessment of the improvement achieved, measure 6 of FIG. 4.

For example, a change in the patients perceived blood loss of about one unit for example from “heavy” to “moderate” or from a score of 3 (“heavy”) to a score of 2 (“moderate”) would provide the basis for continued treatment. While a perceived loss of less than about one unit would suggest either a discontinuation of treatment or a second course after which the evaluation would be reconsidered. Alternatively, or in addition to the blood loss assessment, the practitioner may rely on the assessment in which the comparison of perceived loss is assessed as: i) “about the same”, ii) “better”, and iii) “worse”, as prescribed in measure 6 in FIG. 4. When a patient's response is “about the same”, an alternative treatment regimen may be considered for the next menstrual period. The practitioner may also reconsider re-administering the same treatment regimen for an additional menstrual period and later re-evaluate. When a patient's response is “better”, the assessment may continue by requiring the patient to provide further information about the improvement in menstrual bleeding. For example, the assessment may include “if your menstrual bleeding improved since your last period, please indicate how much” (measure 6b of the MI of FIG. 4). Answers to this inquiry about an improvement in menstrual bleeding may require the patient to provide an answer such as: i) a very great deal better; ii) a great deal better; iii) a good deal better; iv) an average amount better; v) somewhat better; vi) a little better; or vii) almost the same, hardly better at all. Alternatively the answers can be scaled on a seven unit scale where “a very great deal better” is assigned a value of 7 and “almost the same” is valued as 7.

When a patient's response to measure 6 is “worse”, the inquiry continues by requiring the patient to provide further data characterizing the change in menstrual bleeding. For example, the inquiry may determine “if your menstrual period worsened since your last period, please indicate how much” (measure 6c of MI of FIG. 4). Data for this measure to a worsening in menstrual bleeding may require the patient to provide a ranking such as: i) “a very great deal worse”; ii) “a great deal worse”; iii) “a good deal worse”; iv) “an average amount worse”; v) “somewhat worse”; vi) “a little worse”; or vii) “almost the same, hardly worse at all”. As before the answers may be scaled on a seven unit scale where −1 is “almost the same” and −7 is “a very great deal worse”.

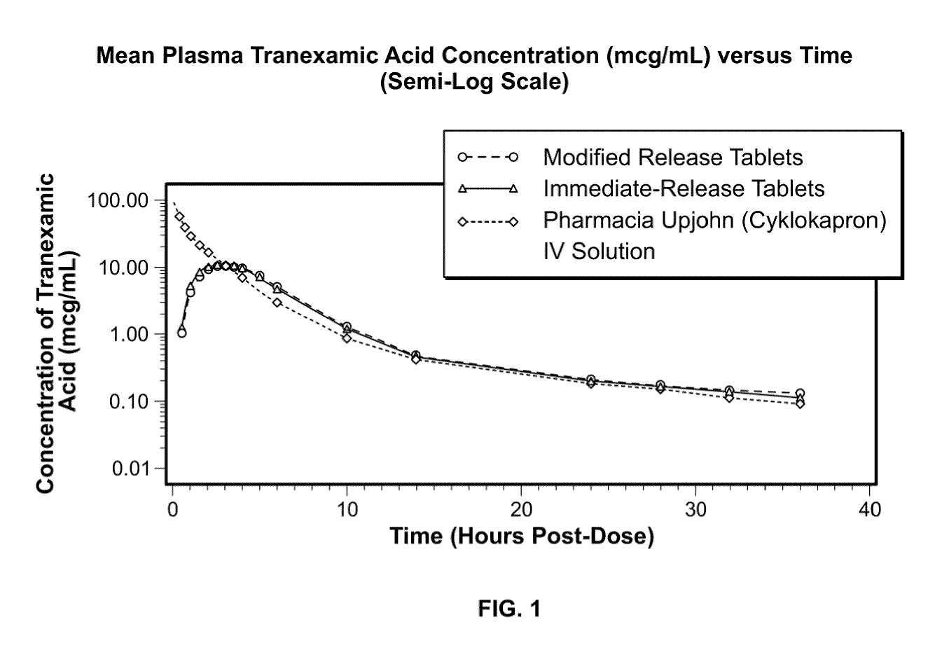

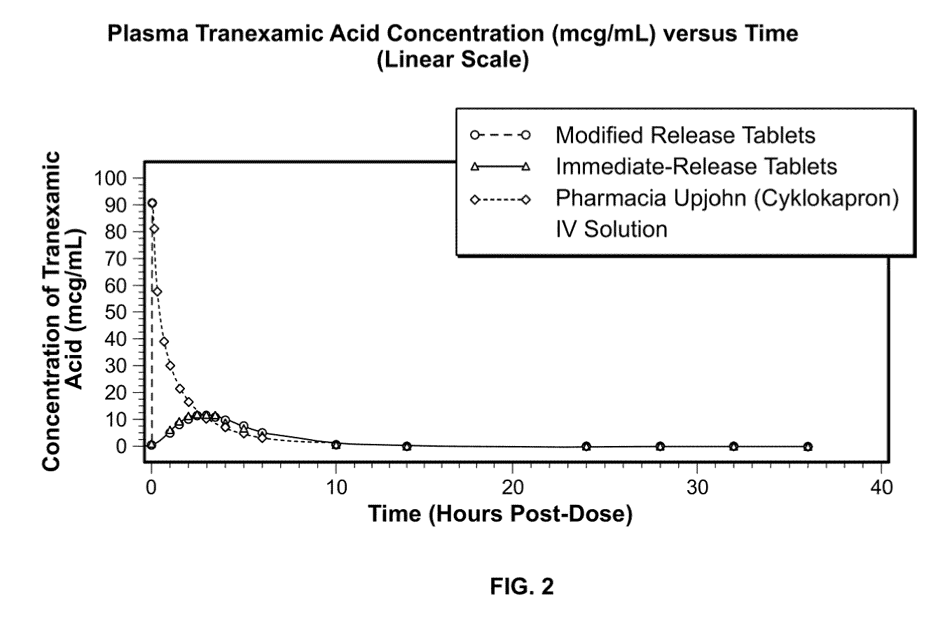

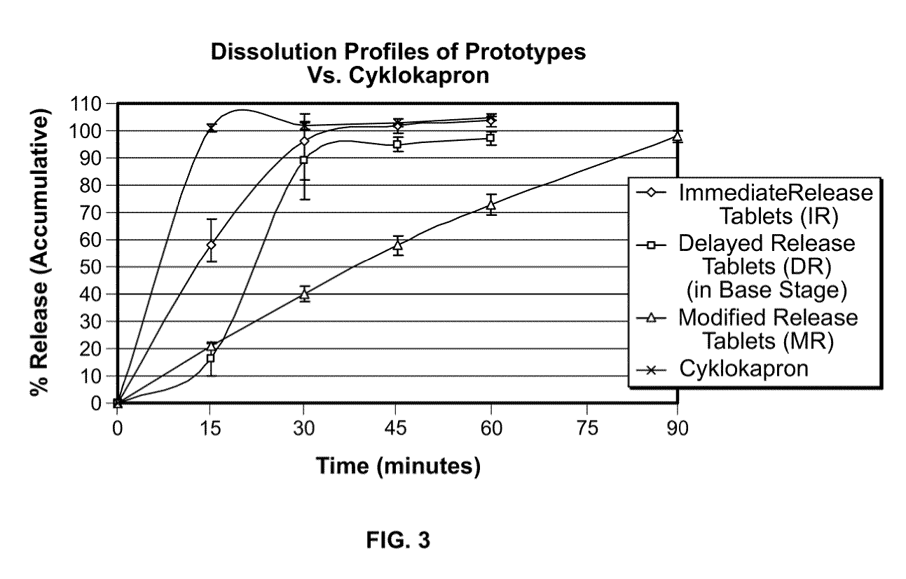

The comparison of perceived blood loss which results in an improvement of at least about one unit as measured by measure 1 of FIG. 4 and/or an assessment of a perceived blood loss which is “better” as provided in measure six of FIG. 1 may proceed by assessing whether the improvement “was a meaningful or an important change” to the patient (measure 6c of MI of FIG. 4).

The information obtained about the “improvement” or “worsening” in menstrual bleeding allows the practitioner to make an evaluation of the effectiveness of the treatment regimen which correlates with the change in blood loss as measured by the alkaline hematin test and demonstrated with clinical trial data.

The method for evaluating the effectiveness of a treatment regimen of the present invention may be repeated after each menstrual period. The data obtained from the initial patient assessment and the subsequent pharmacology (therapy assessment) can be stored into a computer database and utilized for future diagnostic and/or evaluation purposes.

In certain other embodiments, the present invention is directed to a method of treating heavy menstrual bleeding. The method involving, evaluating symptomatic data gathered from the measures individually or collectively as described in FIG. 4. (items one through four and six as discussed above) to determine the need for therapy and then administering, to a patient in need, a therapeutically effective treatment regimen of an antifibrinolytic agent, e.g., a release modified tranexamic acid formulation or a bioequivalent immediate release formulation of the invention, wherein the treatment regimen is to be administered for part or for the duration of menstruation, but no longer than 5 days during the patient's menstrual cycle.

### DETAILED DESCRIPTION OF THE PREFERRED EMBODIMENTS

The invention will be further appreciated with respect to the following examples.

### Example 1

A sustained release formulation includes pH-dependent and -independent binders. Tranexamic acid (5333 g) is combined with methacrylic acid copolymer, Type C (Eudragit® L 100-55 (Rohm Pharma) (200 g), microcrystalline cellulose (Avicel® (142 g), and polyvinyl pyrrolidone powders (20 g) and intimately mixed in a Fielder PMA 65 mixer-granulator. The mixture is granulated with a solution of sodium hydroxide (8 g) in water, and a 30% aqueous dispersion of methyl methacrylate/ethyl acrylate copolymer (Eudragit® NE 30 D (Rohm Pharma) (300 g) is added to the wet mass. The resulting granulate is dried in an Aeromatic Strea-5 fluid bed drier, screened, and then mixed with croscarmellose sodium (10 g) and magnesium stearate (10 g). The mixture is compressed into tablets with a Manesty B tablet press to achieve a dose of 700 mg tranexamic acid per tablet.

### Example 2

A sustained release formulation is prepared according to Example 1 except that Eudragit® L 100-55 is reduced to 100 g, and Eudragit® NE 30 D is replaced by a 40% aqueous dispersion of a methyl methacrylate/ethyl acrylate copolymer (Eudragit® NE 40 D (Rohm Pharma) 200 g).

### Example 3

A sustained release formulation is prepared by blending tranexamic acid 700 mg/tablet with microcrystalline cellulose and polyvinylpyrrolidine K25, granulating with water, drying, and blending with croscarmellose sodium and magnesium stearate. The blend is compressed into tablets and coated with an enteric coating.

### Example 4

An extended release composition is prepared by mixing tranexamic acid (3000 g) and from about 100 g to about 300 g Methocel™ K 100 LV (Dow Chemical Company). The mixture is dry blended, and then is granulated using water until proper granulation is obtained, as known to one skilled in the art. Wet granules are dried in a fluid bed dryer, sifted, and ground to appropriate size. Lubricating and flow agents are mixed with the dried granulation to obtain a final formulation which is compressed into tablets containing 650 mg of tranexamic acid per tablet.

### Example 5

In Example 5, modified release formulations are prepared.

Methocel™ K 100 LV (Dow Chemical Company) is loaded into a mixer and dry blended with tranexamic acid. The mixture is granulated using water until proper granulation is obtained, as known to one skilled in the art. The granulation is then dried, sifted, and ground to appropriate size.

Talc and magnesium stearate are screened and blended with dry granulation. The granulation is loaded into a hopper and compressed into tablets. Tablets are then coated with an aqueous film coating.

In the following formulations, 650 mg tranexamic acid tablets are compressed from the granulation with water added up to the desired quantity (qs) to form the desired granulation then substantially removed by drying.

Formulation one contains 50 mg/tablet Methocel™ K 100 LV Premium CR Grade (Dow Chemical Company), 50 mg/tablet lactose monohydrate, 25 mg/tablet USP talc, and 8 mg/tablet magnesium stearate.

Formulation two contains 75 mg/tablet Methocel™ K 100 LV Premium CR Grade (Dow Chemical Company), 50 mg/tablet lactose monohydrate, 25 mg/tablet USP talc, and 10 mg/tablet magnesium stearate.

Formulation three contains 100 mg/tablet Methocel™ K 100 LV Premium CR Grade (Dow Chemical Company), 50 mg/tablet lactose monohydrate, 30 mg/tablet USP talc, and 10 mg/tablet magnesium stearate.

### Example 6

Modified release 650 mg tranexamic acid tablets were prepared having the ingredients listed in the Table 1 below:

TABLE 1

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose NF (Avicel PH 101) | 5.753 | 44.25 |
| Colloidal Silicon Dioxide NF | 0.0975 | 0.75 |
| Pregelatinized Corn Starch, NF | 6.435 | 49.50 |
| Hypromellose, USP (Methocel K3 Premium LV) | 19.110 | 147.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water USP* | 17.550 | 135.00 |

*Purified water is removed during processing

The formulation of Example 6 was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the hypromellose USP Methocel K3 Premium to the V-blender. Blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets to desired weight.

### Example 7

In Example 7, immediate release 650 mg tranexamic acid tablets were prepared having the ingredients listed in Table 2 below:

TABLE 2

| | Quantity | |
|---|---|---|
| | per batch | Quantity per |
| Ingredient | (kg) | tablet (mg) |
| Active Ingredient | | |
| Tranexamic Acid, EP (650 mg/tab) | 84.50 | 650.0 |
| Inactive Ingredients | | |
| Microcrystalline Cellulose, NF | 5.753 | 44.25 |
| (Avicel PH 101) | | |
| Microcrystalline Cellulose, NF | 10.660 | 82.00 |
| (Avicel PH 102) | | |
| Colloidal Silicon Dioxide, NF | 0.0975 | 0.75 |
| Pregelatinized Corn Starch, NF | 6.435 | 49.50 |
| Croscarmellose Sodium, NF | 1.950 | 15.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water, USP* | 17.550 | 135.00 |
| Film Coating (Inactive Ingredients)** | | |
| Opadry White YS-1-7003 | 4.110 | — |
| Purified Water, USP | 36.990 | — |

*Purified water is removed during processing
**6 kg excess prepared to account for losses during transfer

The formulation of Example 7 was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the croscarmellose sodium and MCC to the V-Blender and blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets.
13. After compression, spray coat the compressed dosage forms with the Opadry White in water.

### Example 8

In Example 8, modified release 650 mg tranexamic acid tablets were prepared as in Example 6 and coated with film coating similar to the immediate release tablets of Example 7. The ingredients are listed in Table 3 below:

TABLE 3

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose NF (Avicel PH 101) | 5.753 | 44.25 |
| Colloidal Silicon Dioxide NF | 0.0975 | 0.75 |
| Pregelatinized Corn Starch, NF | 6.435 | 49.50 |
| Hypromellose, USP (Methocel K3 Premium LV) | 19.110 | 147.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water USP* | 17.550 | 135.00 |
| Film Coating (Inactive Ingredients)** | | |
| Opadry White YS-1-7003 | 4.305 | — |
| Purified Water, USP | 38.750 | — |

*Purified water is removed during processing
**6 kg excess prepared to account for losses during transfer

### Example 9

In Example 9, 650 mg delayed release tranexamic acid tablets were prepared having the ingredients listed in Table 4 below:

TABLE 4

| | Quantity | |
|---|---|---|
| | per batch | Quantity per |
| Ingredient | (kg) | tablet (mg) |
| Active Ingredient | | |
| Tranexamic Acid, EP | 84.50 | 650.0 |
| Inactive Ingredients | | |
| Microcrystalline Cellulose, NF | 5.753 | 44.25 |
| (Avicel PH 101) | | |
| Microcrystalline Cellulose, NF | 10.660 | 82.00 |
| (Avicel PH 102) | | |
| Colloidal Silicon Dioxide, NF | 0.0975 | 0.75 |
| Pregelatinized Corn Starch, NF | 6.435 | 49.50 |
| Croscarmellose Sodium, NF | 1.950 | 15.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water, USP* | 17.550 | 135.00 |
| Film Coating (Inactive Ingredients)** | | |
| Acryl-Eze (93018359) | 12.900 | — |
| Silicone Emulsion, 30% | 0.323 | — |
| Purified Water, USP | 51.271 | — |

*Purified water is removed during processing; mg per tablet is based on a theoretical specific gravity of 1.0 g/ml
**6 kg excess prepared to account for losses during transfer

The formulation of Example 9 was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the croscarmellose sodium and MCC to the V-Blender and blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets.
13. After compression, spray coat the compressed dosage forms with the film coating.

### Example 10

### Delayed Release Tablets with Enteric Coating

In Example 10, 650 mg delayed release tranexamic acid tablets are prepared having the ingredients listed in Table 5 below:

TABLE 5

| | Quantity | | |
|---|---|---|---|
| | per batch | Quantity per | |
| Ingredient | (kg) | tablet (mg) | % |
| Tranexamic Acid Tablets - | | | |
| Quantitative Composition | | | |
| Tranexamic Acid, EP | 84.50 | 650 | 72.2% |
| Microcrystalline Cellulose, NF | 16.41 | 126.25 | 14.0% |
| Colloidal Silicon Dioxide, NF | 0.10 | 0.75 | 0.1% |
| Pregelatinized Corn Starch, NF | 6.44 | 49.5 | 5.5% |
| Croscarmellose Sodium, NF | 1.95 | 15 | 1.7% |
| Povidone, USP | 4.68 | 36 | 4.0% |
| Stearic Acid, NF | 2.34 | 18 | 2.0% |
| Magnesium Stearate, NF | 0.59 | 4.5 | 0.5% |
| Purified Water¹, USP | 17.55 | 135 | |
| Sub-total | 117.00 | 900 | 100.0% |
| Sub-coating composition | | 2 to 3% weight | |
| | | gain | |
| Opadry | | 18 to 27 | |
| Purified Water¹, USP | | — | |
| Enteric Film Coating Composition | | 2 to 3% weight | |
| | | gain | |
| Acryl-Eze | | 180 to 220 | |
| Silicone Emulsion | | 0.2 to 0.3 | |
| Purified Water¹, USP | | | |

¹Purified water is removed during processing.

The formulation of Example 10 is prepared as follows:

Tranexamic acid, pregelatinized corn starch, colloidal silicon dioxide and a portion of the microcrystalline cellulose (5%) are blended in a high shear blender and granulated with a solution of povidone in water. The granulation is dried in a fluid bed drier or a tray drier to a moisture content of approximately 1.2%. The dried granulation is sized through a suitable comminutor such as an oscillating granulator equipped with a 30 mesh screen and added to a V blender. Croscarmellose and the remaining portion (9%) of the microcrystalline cellulose are added to the blender and mixed until uniform. Magnesium stearate and stearic acid are sieved to remove lumps, added to the blender, and blended for approximately 3 minutes. The lubricated granulation is compressed into suitable tablets such as capsule shaped tablets using punches and a dye on a rotary tablet press.

Thereafter, the tablets are coated with a suitable sub-coating to separate the acidic tablet core from the acid sensitive enteric coating using a suitable film forming formulation such as Opadry. Alternatively, comparable sub-coatings may be applied using Spectrablend or a sub-coating formulated with hydroxypropylmethylcellulose and one or more plasticizers. Pigments may be added to the sub-coat to enhance tablet appearance. This coating is typically performed in a rotating coating pan such as an Accelacoater with a spray gun arrangement and heated air.

An enteric coating is then applied in a similar manner as the sub-coating using enteric coating formulations such as Acyl-Eze, Sureteric, or Eudragit L30D, Hydroxypropylmethylcellulose Phthalate (HP-55, Shin-Etsu), Aquateric (FMC), or Eudragit L100-55 (Röhm Pharma). The amount of applied solids needed to protect the tablet from disintegration/dissolution in acid varies with tablet weight and geometry but is typically in the range of about 3 to about 10%.

Alternatively, acceptable tablets can be prepared without a subcoat by applying the enteric film coat directly onto the tablet surface.

### Example 11

### Delayed Release Enteric Coated Tablets Using Fluid Bed Technology

In Example 11, tranexamic acid tablets are prepared in accordance with Example 10 and are coated with an Enteric film coat listed in Table 6 below:

TABLE 6

| Enteric Film | % |
|---|---|
| Coating Composition | |
| Aquateric | 11 |
| Tween 80 | 0.1 |
| Triacetin | 3.9 |
| Purified Water¹, USP | 85 |

¹Purified water is removed during processing.

The coated tranexamic acid tablets of Example 11 are prepared as follows:

A sub-coating of hydroxypropylmethylcellulose can be applied to the tranexamic acid tablets as noted in Example 10. Alternatively, an enteric coating may be applied without a subcoat by film coating directly onto the tablet.

In Example 11, the enteric film coating composition is applied using a fluid bed coating technique with equipment such as a Glatt fluid bed coater configured for film coating or an Aeromatic fluid bed coating. Tablets are charged into the fluid bed coater and suspended in a rapidly moving heated air stream. The coating suspension is sprayed onto the tablets and the water evaporated by the heated air. Additional drying of the coated tablets may be preformed by stopping the spray and continuing the flow of heated air. Monitoring of air flow rates, influent and effluent temperatures, humidity levels, flow rates for the coating solution, and atomizing air pressure assures that this coating process is controlled and reproducible. The amount of applied solids needed to protect the tablet from disintegration/dissolution in acid varies with tablet weight and geometry but is typically in the range of about 3 to about 10%.

### Example 12

### Delayed Release Formulations Utilizing Extrusion-Spheronization

In Example 12 delayed release formulations are prepared. In Example 12, Tranexamic acid pellets or “tiny little time pills” are formulated, enteric coated, and filled into capsules or sachets.

The ingredients for inclusion in the pellets is listed in Table 7 below:

TABLE 7

| | 1 KG Batch | |
|---|---|---|
| Raw Material | Grams | % |
| Tranexamic Acid | 500 | 50% |
| Avicel ® RC-581 | 500 | 50% |
| Purified Water¹ | 1 liter | |
| Total | | 100.0% |

¹Removed during processing

The tranexamic acid pellets of Example 12 are prepared as follows:

Tranexamic acid and Avicel RC-581 are granulated in a high shear granulator with purified water. The wet granulated powders are transferred into an extruder and extruded through a plate or screen to produce small pellets or strings. The pellets/strings are then transferred into a spheronizer which is a chamber with a rotating plate in the bottom. The pellets are spun around for a short period of time which turns the pellets into spheres. The spheres are then dried to a moisture content of about 5%. Spheroids with size ranges between 250μ and 850μ (20/60 mesh size) are obtained by sieving. The spheroids outside of this desired range are discarded or ground and added to future batches. This process produces a very spherical, durable granule. Extrusion/spheronization equipment is produced by Vector/Freund or Niro.

Tranexamic acid spheroids are then coated with enteric coating polymers with or without a sub-coat as described Examples 10 and 11.

The coated spheroids can be filled into gelatin capsules or sachets. The proper dose to be filled in capsules may be determined by the desired capsule size and the fill weight. For example, a 650 mg dose can be obtained by taking two capsules each containing 350 mg of tranexamic acid. Sachet dosing forms are convenient because the desired dose can be added to foods such as applesauce, puddings, juices, drinks, milk, etc.

### Example 13

### Delayed Release Formulation Utilizing Coated Nonpareils

In Example 13 delayed release formulations utilizing beads coated with tranexamic acid are prepared. The ingredients for inclusion in the coated nonpareil beads are listed in Table 8, below.

TABLE 8

| | Quantities used (kg) | | | |
|---|---|---|---|---|
| | Laboratory | Pilot | Semi-ind. | |
| Components | Pan | Pan | Pan | Formula (%) |
| Nonpareils | 0.832 | 6.658 | 46.60 | 42.62 |
| Tranexamic | 1.000 | 8.000 | 56.00 | 51.22 |
| Acid | | | | |
| PVP K30 | 0.048 | 0.382 | 2.70 ± (80%) | 2.45 |
| Talc | 0.072 | 0.580 | 4.06 | 3.71 |

(Ethanol/Purified Water) removed during processing

This coating is performed in a pan with heated air to dry the coating solution. The beads are prepared as follows:

One kilogram of nonpareils having a size of approximately 800 microns is introduced into a laboratory-size solid pan of about 5 to 10 liters. A 20% ethanolic solution of polyvinylpyrrolidone K30 is sprayed onto the non-pareils in order to wet the surface of the nonpareils and make them slightly sticky.

Tranexamic acid is milled and sieved to a particle size of approximately 600 microns. Tranexamic acid powder is dusted onto the bed of sticky nonpareil cores that are rotating in a pan. Talc may be introduced into the bed of cores in the event of sticking. The first cycle is terminated after a short drying period with heated air which allows tranexamic acid to adhere to the core.

The cycles are thus repeated in succession until all of the tranexamic acid is coated onto the cores. To build up the cores properly, about 1/30th of the ethanolic solution and about 1/25th of the tranexamic acid are added at each cycle.

After all of the tranexamic acid has been added, the remaining proportion of the talc is deposited at the surface of the beads with the aid of the solution PVP K30 in order to isolate the tranexamic acid from surface erosion and external treatments.

The beads are further dried in the pan or in a drying oven or fluid bed drier to a low moisture content of approximately 1 to 3%.

This manufacture of tranexamic acid nonpareils can be carried out in a pilot- or semi-industrial-size pan. The procedure remains the same except that the quantities of materials are proportionately increased based on the capacity of the equipment. Care is taken by people skilled in the art to adjust the amount of solution and tranexamic powder added in each cycle so that the beads can be formed without undue sticking, twining, or aggregation and the beads formed are suitable for further processing.

The tranexamic acid beads can be coated, with or without a seal coat, using the formulations and procedures described above for tranexamic acid tablets. Because of the larger surface area for the beads, the total amount of coating added is increased proportionately. Coating amounts of about 20% to about 30% may be added to provide proper protection against dissolution in acidic solutions.

A convenient dosing unit of enteric coated tranexamic acid beads can be obtained by encapsulating the beads in gelatin capsules or packaging the beads in sachets. The proper dose of tranexamic acid to be taken at each dosage period is achieved by proper selection of the capsule size and or the number of capsules to be taken at each dosing period. Sachets containing the desired dose of enteric coated tranexamic acid beads can be added to foods such as applesauce or pudding, sprinkled on salads or other solid foods, or dispersed in juices or milk.

### Example 14

### Adverse Events from Bioavailability and Bioequivalence Evaluation

In Example 14, a comparative, randomized, single dose, 4-way Crossover Absolute Bioavailability (BA) and Bioequivalence (BE) study of Tranexamic Acid Tablet Formulations prepared in accordance with Examples 6 (modified release), 7 (immediate release), and 9 (delayed release) in Healthy Adult Women Volunteers under Fasting Conditions was performed. The design was a randomized, 4-way crossover, comparative BE and BA determination. All oral doses administered were 1.3 g. Twenty-eight (28) healthy non-smoking adult female volunteer subjects were enrolled in the study. Smokers, oral contraceptive users, those with a previous history of thromboembolic events and altered vision were excluded from the study. ECG monitoring was performed before, during and after the estimated times of peak serum tranexamic acid concentrations exposure. During the study, adverse events were captured and recorded throughout the trial period.

In the study, subjects were randomized to receive single oral 1.3 g (2×650 mg tablets) dose of tranexamic acid in tablet forms which included a modified release dosage form, an immediate release dosage form, and a delayed release dosage form. Subjects were also administered a single 1 g (10 ml) IV solution of tranexamic acid (100 mg/ml concentration).

The most frequently reported adverse events (Reported by greater than 10% of subjects) from the study of Example 14 are listed in the table below. The table lists the number of subjects reporting adverse events, and the percentage of subjects is in parentheses.

TABLE 9
Most Frequently Reported Adverse Events
(Reported by >10% of Subjects)
Number of Subjects Reporting Adverse Events

| | Treatment | | | | |
|---|---|---|---|---|---|
| | Delayed | Modified | Immediate | IV | |
| | Release | Release | Release | solution | |
| | (2 × | (2 × | (2 × | (10 × 100 | |
| Adverse | 650 mg) | 650 mg) | 650 mg) | mg/ml) | Total |
| Events | (n = 27) | (n = 27) | (n = 27) | (n = 27) | (n = 28) |
| Headache | 6 (22%) | 4 (15%) | 7 (26%) | 7 (26%) | 14 (50%) |
| Nausea | 0 (0%) | 0 (0%) | 2 (7%) | 10 (37%) | 12 (43%) |
| Dizziness | 1 (4%) | 0 (0%) | 0 (0%) | 11 (41%) | 12 (43%) |
| Feeling | 0 (0%) | 0 (0%) | 0 (0%) | 6 (22%) | 6 (21%) |
| Hot | | | | | |
| Nasal | 1 (4%) | 2 (7%) | 1 (4%) | 1 (4%) | 5 (18%) |
| Congestion | | | | | |
| Cough | 1 (4%) | 0 (0%) | 0 (0%) | 2 (7%) | 3 (11%) |
| Urine odor | 2 (7%) | 2 (7%) | 0 (0%) | 1 (4%) | 3 (11%) |
| abnormal | | | | | |

### Example 15

Dissolution tests of Modified Release and Immediate Release Formulations prepared in accordance with Examples 6 and 7 respectively were performed under USP 27 Dissolution Apparatus Type II Paddle Method @ 50 RPM in 900 ml water at 37±0.5° C. The delayed release formulation of Example 9 was dissolution tested under USP 27 Dissolution Apparatus Type II Paddle Method @ 50 RPM and 37±0.5° C. for 120 minutes in acid medium (1000 ml of 0.1N hydrochloric acid), subsequently followed by buffer medium (1000 ml of pH 6.8 phosphate buffer) for 45 minutes.

The results are listed in the tables below.

TABLE 10
Dissolution Results for the Immediate Release Formulation.

| Time (min.) | Dissolution (%) | Standard Deviation |
|---|---|---|
| 15 | 58.0% | ±9.521905 |
| 30 | 96.0% | ±10.2697 |
| 45 | 102.0% | ±0.408248 |
| 60 | 104.0% | ±1.032796 |

TABLE 11
Dissolution Results for the Modified Release Formulation

| Time (min.) | Dissolution (%) | Standard Deviation |
|---|---|---|
| 15 | 21.0% | ±1.414214 |
| 30 | 40.0% | ±2.810694 |
| 45 | 58.0% | ±3.600926 |
| 60 | 73.0% | ±3.81663 |
| 90 | 98.0% | ±2.097618 |

The ratios of least-squares means and the 90% confidence intervals derived from the analyses of the In-transformed pharmacokinetic parameters AUC₀₋ₜ, AUC_{inf} and Cₘₐₓ for tranexamic acid in plasma were within the 80-125% Food and Drug Administration (FDA) acceptance range for the modified release formulation versus the immediate release formulation under fasting conditions.

The absolute bioavailability of the modified release and immediate release tablet formulations were 44.93% and 46.04% respectively

Based on these results, the modified release tranexamic acid tablet formulation and the immediate release tranexamic acid formulation are bioequivalent under fasting conditions.

TABLE 12
Dissolution Results for the Delayed Release Formulation
(in base stage)

| Time (min.) | Dissolution (%) | Standard Deviation |
|---|---|---|
| 15 | 16% | ±6.013873 |
| 30 | 89% | ±14.06769 |
| 45 | 95% | ±2.810694 |
| 60 | 97% | ±2.345208 |

### Example 16

### Comparative Example

In Comparative Example 16, a 500 mg immediate release tranexamic acid tablet, approved and marketed in Canada under the name Cyklokapron was obtained and dissolution tested under USP 27 Apparatus Type II Paddle Method @ 50 RPM in 900 ml water at 37±0.5° C. The dissolution results are listed in Table 13 below:

TABLE 13

| | % dissolved | % dissolved | % dissolve | % dissolved |
|---|---|---|---|---|
| Sample # | in 15 min. | in 30 min. | in 45 min. | in 60 min. |
| 1 | 102 | 104 | 105 | 106 |
| 2 | 102 | 104 | 105 | 106 |
| 3 | 101 | 102 | 102 | 105 |
| 4 | 99 | 101 | 102 | 103 |
| 5 | 100 | 102 | 103 | 104 |
| 6 | 99 | 101 | 102 | 104 |
| Average | 101 | 102 | 103 | 105 |
| % RSD | 1.4 | 1.3 | 1.4 | 1.1 |

### Example 17

### Mennoraghia Impact Measure Validation

Objective measurements of menstrual blood loss are not practical in the healthcare setting, and they correlate poorly with a woman's subjective assessment of blood loss and its impact on quality of life [Warner 2004; National Collaborating Centre for Women's and Children's Health, 2007]. Menorrhagia is a subjective condition and may be practically defined as menstrual loss that is greater than the woman feels that she can reasonably manage. The amelioration of symptoms of heavy menstrual loss are practical efficacy benefits of the treatment are therefore important to measure and validate in a controlled clinical environment.

The MI was evaluated in a sub population of patients enrolled in a clinical trial designed to assess the safety and efficacy of modified release tranexamic acid formulation (Example 2) at an oral dose of 3.9 g administered daily for up to 5 days during each menstrual period (While the present study was conducted utilizing the modified release tranexamic acid formulation described herein in Example 2, the effectiveness of the MI can also be evaluated utilizing the immediate release and delayed release formulations described herein). Two groups of patients were used to assess the MI, one group of patients were those diagnosed with menorrhagia and undergoing treatment. The second group was an age matched normal group. The sub-study was designed: to collect sufficient quantitative data to support the construct-related validation of the MI measures; to collect sufficient quantitative data to support the assessment of meaningful/important change in blood loss to the women; to conduct a test/retest evaluation of the instrument, and to address the reliability of the MI measures.

### Study Methods

Development of the MI began with a review of the literature focusing on the methods used to collect qualitative data from menorrhagia patients. Qualitative interviews with patients determined which symptomatic concepts were most important to women and could be included in a draft Impact Measure. Cognitive debriefing interviews to evaluate patient understanding of items led to the synthesis of a patient-based instrument for assessing the impact of limitations caused by heavy menstrual bleeding. Published measures were used in the evaluation of the psychometric properties of the Menorrhagia Instrument to assess Construct-Related Validity. The reference measures include, the Ruta Menorrhagia Questionnaire [Ruta 1995] and the Medical Outcomes Study Short-Form 36 Item Health Status Instrument (SF-36) [Ware 1992]. Scoring of the standardized measures followed published algorithms, Table 14.

TABLE 14
Descriptions of Instruments used in this study

| Measure | Score Generated | Score Ranges |
|---|---|---|
| Menorrhagia Impact | Blood Loss Severity (Q1) | 1 (light) thru 4 (very heavy) |
| Measure (MI) | Limitation | |
| | Work outside or inside the home (Q2) | 1 (not at all) thru 5 (extremely) |
| | Physical activities (Q3) | 1 (not at all) thru 5 (extremely) |
| | Social or leisure activities (Q4) | 1 (not at all) thru 5 (extremely) |
| | Activity list (Q5) | [Descriptive] |
| | Change in blood loss (follow-up) (Q6, 6a, 6b) | [15-pt scale: 0 = no change, |
| | | 1-7 improve, 1-7 worse] |
| | Meaningful/important change (Q6c) | Y/N |
| Ruta Menorrhagia | Global | 0 (asymptomatic) - 42 (severe) |
| Questionnaire | Specific | |
| | Physical Function: Impact on work and daily | 0 (asymptomatic) - 6 (severe) |
| | activities (Q9 and Q10) | |
| | Social Function: Impact on social and leisure | 0 (asymptomatic) - 8 (severe) |
| | activities and sex-life (Q11 and Q12) | |
| SF-36 | Physical Functioning, Role-Physical, Bodily Pain | 0-100 |
| | General Health (can be combined to form Physical | (100 = minimal impairment) |
| | Health Component Score); Vitality, Social | |
| | Functioning, Role-Emotional, Mental Health (can | |
| | be combined to form Mental Health Component | |
| | Score) | |

### Study Design

A total of 262 women completed the MI. The MI measures 1 through 5 were administered after subject's baseline period and after the subsequent first, second, third and sixth treatment periods. The MI measure 6 was administered after the first treatment period only. For this validation study, only the data collected through Month 1 of treatment was included in the analyses for the treatment cohort. The MI measures 1-5 were administered at baseline and at the subsequent first and second non-treatment periods for the subjects in the normal cohort The MI measure 6 was administered and data collected, at Month 1 and Month 2. The Ruta Menorrhagia Questionnaire, SF-36 Health Survey and the MI were completed by the subject before visit procedures were performed. A subset of at least 50 subjects were asked to return to the study site 7 to 10 days after the baseline Visit but before the next menstrual period starts to complete the MI a second time.

### Treatment Group

A total of 177 patients were enrolled into the sub-study. During this time period 28 patients withdrew consent, dropped-out, or did not properly complete MI and were non-evaluable. The 149 patients remaining were intended to be age matched. The majority of patients in the study were in their late 30's or early 40's. Because of the difficulty of enrolling sufficient numbers of women with normal menstrual periods in this age bracket 18 evaluable patients were not age matched. A total of 131 evaluable patients were age matched. A sub-set of 80 evaluable patients participated in the test/retest segment of the validation. Of these patients 11 were evaluable but not age matched. Data from all 80 patients were used for statistical evaluation of the test/re-test correlations.

### Normal Group

A group of women with self reported normal menstrual bleeding comprised the pool of normal women eligible for age matching in the study. A normal was defined as all of the following: a menstrual cycle between 26 and 32 days long, and their last (most recently completed) menstrual period was seven days or less in duration, the heaviest bleeding was three days or less, and the woman classified the bleeding overall as “light” or “moderate” as opposed to “heavy” or “very heavy. Women with normal periods who were enrolled into the study served as age-match controls for women recruited into the treatment group. Un-matching and re-matching occurred throughout the enrollment period if participants in either group dropped out of the study, if better re-matching increased the total number of matched pairs, or if the age-matched woman with normal periods did not enroll in the study.

Five women enrolled in the study did not complete the study through Visit 3. Another five women who did complete the study became ‘unmatched’ as the Treatment Group participant they had been matched to became non-evaluable. The 131 women who completed the study and remained matched are the Validation Sample Normal Group. A total of 51 women completed the Retest.

The following Measures were summarized and statistically analyzed:

MI measure 1—Blood Loss Rating

MI measure 2—Limitation of Work Outside or Inside the Home

MI measure 3—Limitation of Physical Activities

MI measure 4—Limitation of Social or Leisure Activities

MI measure 6/6a/6b—Menstrual Blood Loss During Last Period

MI measure 6c—Meaningfulness of Change in Menstrual Blood Loss

The statistics include the counts (missing data), mean, standard deviation, median, inter-quartile range, and minimum/maximum values. Differences in these variables between the treatment and normal cohorts were assessed using analysis of variance.

A p-value<0.05 was required for significance using two-sided hypothesis tests; no p-value adjustments were made for the analysis of multiple endpoints. All analyses were performed under SPSS version 11.5 for Windows, and the Stuart-Maxwell test for homogeneity was performed using Stata version 9.0 for Windows.

Validation of the MI was conducted using standardized analytic procedures found in the FDA Draft Guidance on Patient Reported Outcomes for Use in Evaluating Medical Products for Labeling Claims and instrument review criteria developed by the Scientific Advisory Committee of the Medical Outcomes Trust.¹ ¹ Scientific Advisory Committee of the Medical Outcomes Trust. Assessing health status and quality-of-life instruments: attributes and review criteria. Qual Life Res. 2002; 11: 193-205

### Evaluation of the Menorrhagia Instrument

The MI consisted of 4 individual measures (1-4) that were analyzed separately for validation. No summative scale was derived. Measure 5, served as descriptive of variables and did not undergo standard validation analyses. Measures 6, 6a and 6b dealt with menstrual blood loss relative to the previous menstrual period. The answers to the measures in the subparts of measure 6, were combined to produce a 15 point rating scale. The scale values range from −7 to +7 with −7 representing a very great deal worse menstrual blood loss than the previous period, and +7 representing a very great deal better menstrual blood loss than the previous period. The midpoint (0) represents the perception of about the same menstrual blood loss as the previous period.

Test-retest reliability assessed if items produced stable, reliable scores under similar conditions (Guttman, 1945). Reproducibility was evaluated in a subset of at least 50 from the treatment group and at least 50 from the normal group 7 to 10 days after the baseline visit using the intra-class correlation coefficient (ICC, see formula below). Values above 0.70 indicated the stability of an instrument over time. The following formula was used to compute the Intraclass Correlation Coefficient (ICC):

$$ICC = \frac{{A}^{2} + {B}^{2} + {C}^{2}}{{A}^{2} + {B}^{2} + {D}^{2} - ( \frac{{C}^{2}}{n} )}$$

where:
A=Standard deviation of baseline score
B=Standard deviation of Time 2 score
C=Standard deviation of change in score
D=mean of change in score
n=number of respondents

The data for each of the measures was above 0.70. In the test population, n=88, values of 0.72 (0.60-0.81), 0.75 (0.64-0.83), 0.77 (0.67-0.84) and 0.76 (0.66-0.84) for measures 1 to 4 respectively. The aged matched normal values where n=51 were 0.77 (0.63-0.86), 0.67 (0.49-0.80), 0.75 (0.60-0.85) and 0.86 (0.77-0.92) respectively.

Construct-Related Validity was established when relationships among items, domains, and concepts conform to what was predicted by the conceptual framework for the instrument. This includes convergent, discriminant, and known-groups validity. Convergent and discriminant validity was present where measures of the same construct are more highly related and measures of different constructs were less related. To assess convergent and discriminant validity, Pearson's correlation coefficients were computed between each MI measure and items and scales from the SF-36 and the Ruta Menorrhagia Questionnaire included in the study design and administered at the same visit. The following hypotheses were tested:

The MI Blood Loss Measure (#1) will have a stronger association with the Ruta Menorrhagia Questionnaire (RMQ) than to the SF-36 subscales.

The MI Physical Activity Limitation Measure (#3) will have a stronger association with the RMQ Physical Function scale, the SF-36 Physical domain, the SF-36 Role-Physical domain, and SF-36 Physical Component Summary score than the Ruta Social, SF-36 Social, and SF-36 Vitality domains.

The MI Social/Leisure Activity Limitation will have a have stronger associations with the RMQ Social Function scale and the SF-36 Social Function domain than the RMQ Physical, the SF-36 Physical and SF-36 Bodily Pain domains.

For convergent validity, the correlations of MI measures with Ruta subscales, SF-36 subscales, and diary data are shown in Table 24. The Ruta global score was highly correlated with each MI measures (range 0.757-0.809). The correlations of items with the SF-36 subscales were low to moderate, which is to be expected since the SF-36 is not a disease-specific measure, but rather a more generic health status measure unable to detect differences between a normal population and a population of women with menorrhagia. The MI measures were more strongly correlated with the SF-36 Physical and Role Physical subscales than other SF-36 subscales.

TABLE 15
Correlations Between Menorrhagia Insrtument Patient Reported
Outcome (PRO) Measures and Ruta/SF-36/Diary

| | | | | MI measure 4 |
|---|---|---|---|---|
| | MI measure 1 | MI measure 2 | MI measure 3 | Limit social or |
| | Blood | Limit work outside or | Limit physical | leisure |
| | Loss | inside home | activity | activity |
| Ruta - Global | 0.767 | 0.785 (0.000) | 0.807 (0.000) | 0.809 (0.000) |
| | (0.000) | | | |
| Ruta - | 0.512 | 0.682 (0.000) | 0.646 (0.000) | 0.664 (0.000) |
| Physical Fx | (0.000) | | | |
| Ruta - Social | 0.606 | 0.634 (0.000) | 0.659 (0.000) | 0.683 (0.000) |
| Fx | (0.000) | | | |
| SF-36 - | −0.229 | −0.234 (0.000) | −0.264 (0.000) | −0.273 (0.000) |
| Physical Fx | (0.000) | | | |
| SF-36 - | −0.118 | −0.194 (0.002) | −0.200 (0.001) | −0.261 (0.000) |
| Social Fx | (0.057) | | | |
| SF-36 - Role | −0.200 | −0.279 (0.000) | −0.258 (0.000) | −0.303 (0.000) |
| Physical | (0.001) | | | |
| SF-36 - | −0.143 | −0.193 (0.002) | −0.248 (0.000) | −0.250 (0.000) |
| Vitality | (0.021) | | | |
| SF-36 - | −0.087 | −0.168 (0.006) | −0.192 (0.002) | −0.205 (0.001) |
| Bodily Pain | (0.163) | | | |
| SF-36 -PCS | −0.190 | −0.271 (0.000) | −0.285 (0.000) | −0.275 (0.000) |
| | (0.002) | | | |

The data supported the hypothesis that the MI Blood Loss measure (#1) had a stronger association with the Ruta global score than to the SF-36 subscales. While the hypothesis that MI measure #3 (Physical Activity Limitation) would be strongly associated to the physical domains of the RMQ (r=0.65) and SF-36 (r=−0.26) was confirmed, this measure was also strongly correlated to the RMQ Social Functioning (r=0.66). MI measure #4 (Social or Leisure Activity Limitation) was highly correlated to the RMQ Social (r=0.68) and moderately associated with the SF-36 Social Functioning domain.

Known-groups validity determined the ability of the instrument to discriminate between groups of subjects known to be distinct. The ability of the MI items to discriminate among known groups was assessed by comparing the 4 items (1 thru 4) to responses from the two groups (treatment and normal) at baseline. Differences in these variables, between the treatment and normal groups, were assessed using analysis of variance. A p-value<0.05 was required for significance using two-sided hypothesis tests; no p-value adjustments was made for the analysis of multiple endpoints.

For each MI measure, the mean score for the treatment group was significantly different than the mean score for the normal group (p<0.001). The treatment group scores were higher for each individual measure, indicating greater limitation as a result of their excessive menstrual blood loss (see Table 16).

TABLE 16
Known-Groups Validity of the MI

| | | | | | AGE MATCH | | | |
|---|---|---|---|---|---|---|---|---|
| | | Treatment | | | NORMAL | | | |
| | | Cohort | | | Cohort | | | |
| | | N | Mean | St. Dev. | N | Mean | St. Dev. | F (sig.)¹ |
| MI | Self- | 131 | 3.25 | 0.61 | 131 | 2.10 | 0.61 | 234.727 |
| measure 1 | perceived | | | | | | | (<0.001) |
| | blood loss | | | | | | | |
| MI | Limit you in | 131 | 3.04 | 0.99 | 131 | 1.34 | 0.59 | 286.864 |
| measure 2 | your work | | | | | | | (<0.001) |
| MI | Limit you in | 131 | 3.28 | 0.95 | 131 | 1.49 | 0.72 | 299.011 |
| measure 3 | your physical | | | | | | | (<0.001) |
| | activities | | | | | | | |
| MI | Limit you in | 131 | 3.05 | 1.06 | 131 | 1.37 | 0.72 | 227.312 |
| measure 4 | your | | | | | | | (<0.001) |
| | social/leisure | | | | | | | |
| | activities | | | | | | | |

The ability to detect change required that values for the item or instrument change when the concept it measures changed. In order to measure the MI items ability to detect change, longitudinal data were evaluated focusing primarily on the changes from baseline to month 1. Differences in proportions and comparisons between treatment and normal groups were compared using chi-square statistics (the Stuart-Maxwell test testing marginal homogeneity for all categories simultaneously). Cohen Effect Size statistics were also compared between the treatment and normal groups. The Cohen Effect Size was computed by taking the mean change in measure score (baseline to month 1) and dividing that by the standard deviation of mean baseline score². ² Cohen, J. J. (1988). Statistical power analysis for the behavioral sciences (p. 8). Erlbaum: Hillsdale, N.J.

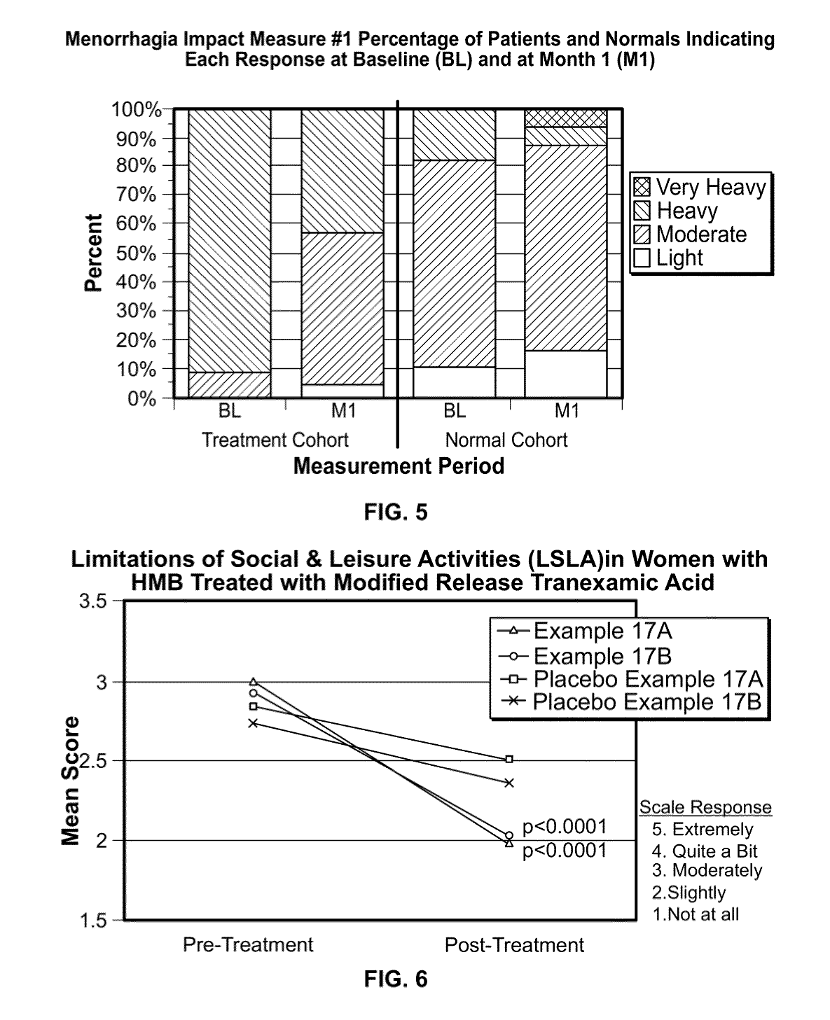

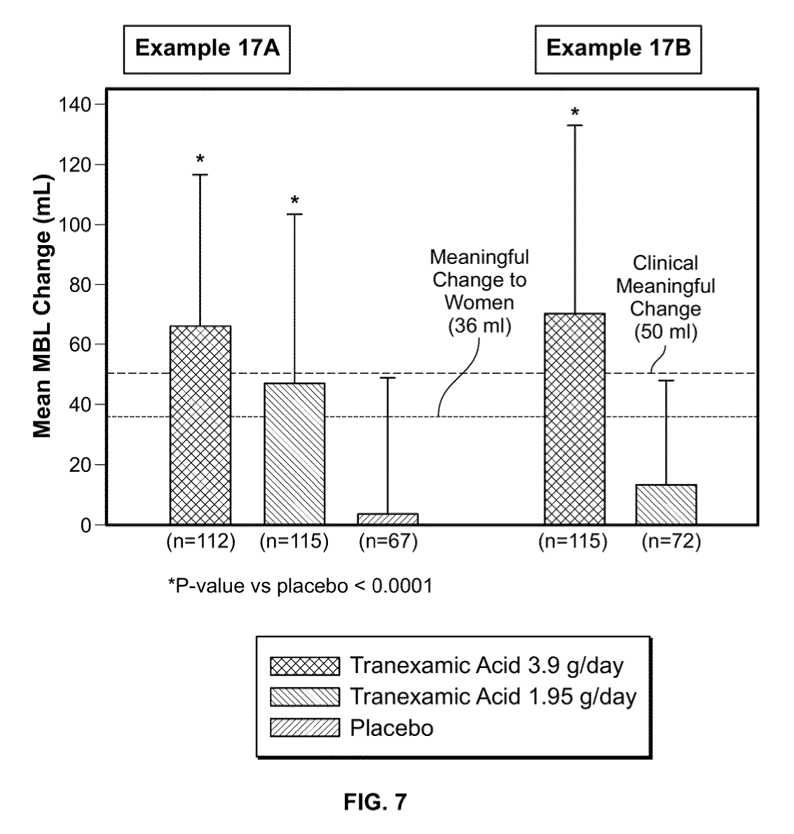

Ability to detect change was described for each item in Tables 17A-D by indicating the distribution of baseline and month 1 response option pairs for all patients. Change in responses from baseline to month 1 was tested using the Stuart-Maxwell test. For the treatment group, there was significant change in responses to each measure from baseline to month one (p<0.001). For the normal group, none of the items had significant changes in responses from baseline to month one. FIG. 5 illustrates the distribution of responses to measure 1 at baseline and at month one. In the treatment group, the proportion reporting light or moderate bleeding as measured with item 1, increased from baseline to month 1, and in the normal group this proportion changed very little.

TABLE 17A
Sensitivity to change of the MI Measure 1

| | | | Month 1 | | | | |
|---|---|---|---|---|---|---|---|
| | | Response | | | | Very | Stuart-Maxwell |
| Cohort | | category | Light | Moderate | Heavy | Heavy | test of association |
| Treatment | Baseline | Light | 0 | 0 | 0 | 0 | 59.09 (p < 0.001) |
| | | | (0.0%) | (0.0%) | (0.0%) | (0.0%) | |
| | | Moderate | 0 | 8 | 4 | 0 | |
| | | | (0.0%) | (6.3%) | (3.2%) | (0.0%) | |
| | | Heavy | 3 | 41 | 24 | 2 | |
| | | | (2.4%) | (32.5%) | (19.0%) | (1.6%) | |
| | | Very Heavy | 2 | 18 | 13 | 11 | |
| | | | (1.6%) | (14.3%) | (10.3%) | (8.7%) | |
| Normal | Baseline | Light | 9 | 5 | 0 | 0 | 6.35 (p = 0.130) |
| | | | (6.9%) | (3.8%) | (0.0%) | (0.0%) | |
| | | Moderate | 12 | 77 | 4 | 0 | |
| | | | (9.2%) | (59.2%) | (3.1%) | (0.0%) | |
| | | Heavy | 0 | 9 | 8 | 2 | |
| | | | (0.0%) | (6.9%) | (6.2%) | (1.5%) | |
| | | Very Heavy | 0 | 2 | 2 | 0 | |
| | | | (0.0%) | (1.5%) | (1.5%) | (0.0%) | |

TABLE 17B
Sensitivity to change of the MI Measure 2

| | | | Month 1 | | | | | |
|---|---|---|---|---|---|---|---|---|
| | | | | | | | | Stuart- |
| | | | | | | | | Maxwell |
| | | Response | Not at | | | Quite | | test of |
| Cohort | | category | all | Slightly | Moderately | a bit | Extremely | association |
| Treatment | Baseline | Not at all | 5 | 0 | 1 (0.8%) | 1 | 0 (0.0%) | 53.33 |
| | | | (4.0%) | (0.0%) | | (0.8%) | | (p < 0.001) |
| | | Slightly | 12 | 11 | 2 (1.6%) | 1 | 0 (0.0%) | |
| | | | (9.5%) | (8.7%) | | (0.8%) | | |
| | | Moderately | 17 | 26 | 14 (11.1%) | 1 | 0 (0.0%) | |
| | | | (13.5%) | (20.6%) | | (0.8%) | | |
| | | Quite a bit | 2 | 8 | 5 (4.0%) | 9 | 0 (0.0%) | |
| | | | (1.6%) | (6.3%) | | (7.1%) | | |
| | | Extremely | 3 | 3 | 3 (2.4%) | 1 | 1 (0.8%) | |
| | | | (2.4%) | (2.4%) | | (0.8%) | | |
| Normal | Baseline | Not at all | 89 | 5 | 1 (0.8%) | 0 | 0 (0.0%) | 2.86 |
| | | | (69.0%) | (3.9%) | | (0.0%) | | (p = 0.517) |
| | | Slightly | 8 | 13 | 4 (3.1%) | 2 | 0 (0.0%) | |
| | | | (6.2%) | (10.1%) | | (1.6%) | | |
| | | Moderately | 0 | 3 | 4 (3.1%) | 0 | 0 (0.0%) | |
| | | | (0.0%) | (2.3%) | | (0.0%) | | |
| | | Quite a bit | 0 | 0 | 0 (0.0%) | 0 | 0 (0.0%) | |
| | | | (0.0%) | (0.0%) | | (0.0%) | | |
| | | Extremely | 0 | 0 | 0 (0.0%) | 0 | 0 (0.0%) | |
| | | | (0.0%) | (0.0%) | | (0.0%) | | |

TABLE 17C
Sensitivity to change of the MI Measure 3

| | | | | | | | | Stuart- |
|---|---|---|---|---|---|---|---|---|
| | | | Month 1 | | | | | Maxwell |
| | | Response | Not at | | | Quite | | test of |
| Cohort | | category | all | Slightly | Moderately | a bit | Extremely | association |
| Treatment | Baseline | Not at all | 0 | 0 | 1 (0.8%) | 0 | 0 (0.0%) | 64.58 |
| | | | (0.0%) | (0.0%) | | (0.0%) | | (p < 0.001) |
| | | Slightly | 12 | 12 | 1 (0.8%) | 1 | 0 (0.0%) | |
| | | | (9.5%) | (9.5%) | | (0.8%) | | |
| | | Moderately | 14 | 20 | 11 (8.7%) | 3 | 0 (0.0%) | |
| | | | (11.1%) | (15.9%) | | (2.4%) | | |
| | | Quite a bit | 6 | 17 | 9 (7.1%) | 5 | 0 (0.0%) | |
| | | | (4.8%) | (13.5%) | | (4.0%) | | |
| | | Extremely | 5 | 2 | 2 (1.6%) | 3 | 2 (1.6%) | |
| | | | (4.0%) | (1.6%) | | (2.4%) | | |
| Normal | Baseline | Not at all | 72 | 9 | 0 (0.0%) | 0 | 0 (0.0%) | 1.99 |
| | | | (55.4%) | (6.9%) | | (0.0%) | | (p = 0.708) |
| | | Slightly | 14 | 18 | 3 (2.3%) | 1 | 0 (0.0%) | |
| | | | (10.8%) | (13.8%) | | (0.8%) | | |
| | | Moderately | 0 | 6 | 4 (3.1%) | 1 | 0 (0.0%) | |
| | | | (0.0%) | (4.6%) | | (0.8%) | | |
| | | Quite a bit | 0 | 1 | 1 (0.8%) | 0 | 0 (0.0%) | |
| | | | (0.0%) | (0.8%) | | (0.0%) | | |
| | | Extremely | 0 | 0 | 0 (0.0%) | 0 | 0 (0.0%) | |
| | | | (0.0%) | (0.0%) | | (0.0%) | | |

TABLE 17D
Sensitivity to change of the MI Measure 4

| | | | | | | | | Stuart- |
|---|---|---|---|---|---|---|---|---|
| | | | Month 1 | | | | | Maxwell |
| | | Response | Not at | | | Quite | | test of |
| Cohort | | category | all | Slightly | Moderately | a bit | Extremely | association |
| Treatment | Baseline | Not at all | 6 | 3 | 0 (0.0%) | 0 | 0 (0.0%) | 60.77 |
| | | | (4.8%) | (2.4%) | | (0.0%) | | (p < 0.001) |
| | | Slightly | 16 | 10 | 0 (0.0%) | 2 | 0 (0.0%) | |
| | | | (12.7%) | (7.9%) | | (1.6%) | | |
| | | Moderately | 19 | 14 | 12 (9.5%) | 2 | 1 (0.8%) | |
| | | | (15.1%) | (11.1%) | | (1.6%) | | |
| | | Quite a bit | 5 | 14 | 4 (3.2%) | 6 | 0 (0.0%) | |
| | | | (4.0%) | (11.1%) | | (4.8%) | | |
| | | Extremely | 3 | 4 | 1 (0.8%) | 3 | 1 (0.8%) | |
| | | | (2.4%) | (3.2%) | | (2.4%) | | |
| Normal | Baseline | Not at all | 84 | 11 | 0 (0.0%) | 0 | 0 (0.0%) | 1.71 |
| | | | (64.6%) | (8.5%) | | (0.0%) | | (p = 0.807) |
| | | Slightly | 10 | 14 | 2 (1.5%) | 0 | 0 (0.0%) | |
| | | | (7.7%) | (10.8%) | | (0.0%) | | |
| | | Moderately | 0 | 4 | 2 (1.5%) | 0 | 0 (0.0%) | |
| | | | (0.0%) | (3.1%) | | (0.0%) | | |
| | | Quite a bit | 0 | 0 | 0 (0.0%) | 2 | 0 (0.0%) | |
| | | | (0.0%) | (0.0%) | | (1.5%) | | |
| | | Extremely | 1 | 0 | 0 (0.0%) | 0 | 0 (0.0%) | |
| | | | (0.8%) | (0.0%) | | (0.0%) | | |

The amount of change in each item from baseline to month 1 is shown in Table 18. For the treatment group, the mean change in response from baseline to month 1 ranged from −0.76 to −1.16 for the four items. The calculated effect size shows this amount of change for each item ranged from −0.9 to −1.2. For the normal group, the mean change in response from baseline to month 1 ranged from 0.03 to −0.12 for the four items. The effect size for each item ranged from 0.053 to −0.197. This analysis shows a large response in patients undergoing treatment and little to no response in normal women who have received no treatment. This instrument is capable of identifying the perceived improvement in menstrual blood loss.

TABLE 18
Sensitivity to Change of MI Effect Size

| | | BASELINE | | | | | | CHANGE | | | |
|---|---|---|---|---|---|---|---|---|---|---|---|
| | | | | St | | | St | | | St | Effect |
| Menorrhagia Item | | n | Mean | Dev | n | Mean | Dev | n | Mean | Dev | Size¹ |
| | | | | | MONTH 1 | | | | | | |
| Item 1 | Self-perceived blood | 126 | 3.25 | 0.62 | 126 | 2.49 | 0.73 | 126 | −0.76 | 0.84 | −1.226 |
| | loss | | | | | | | | | | |
| Item 2 | Limit you in your work | 126 | 3.05 | 0.99 | 126 | 2.12 | 0.99 | 126 | −0.93 | 1.13 | −0.939 |
| Item 3 | Limit you in your | 126 | 3.29 | 0.95 | 126 | 2.13 | 1.00 | 126 | −1.16 | 1.17 | −1.221 |
| | physical activities | | | | | | | | | | |
| Item 4 | Limit you in your | 126 | 3.06 | 1.06 | 126 | 2.00 | 1.04 | 126 | −1.06 | 1.19 | −1.000 |
| | social/leisure activities | | | | | | | | | | |
| | | | | | CHANGE | | | | | | |
| Item 1 | Self-perceived blood | 130 | 2.10 | 0.61 | 130 | 1.98 | | 130 | −0.12 | 0.56 | −0.197 |
| | loss | | | | | | | | | | |
| Item 2 | Limit you in your work | 129 | 1.32 | 0.57 | 129 | 1.35 | | 129 | 0.03 | 0.50 | 0.053 |
| Item 3 | Limit you in your | 130 | 1.49 | 0.72 | 130 | 1.43 | | 130 | −0.06 | 0.57 | −0.083 |
| | physical activities | | | | | | | | | | |
| Item 4 | Limit you in your | 130 | 1.37 | 0.72 | 130 | 1.33 | | 130 | −0.04 | 0.58 | −0.056 |
| | social/leisure activities | | | | | | | | | | |

Responses from treatment group participants were divided based on two separate responder definitions. In the first definition, a responder was a patient indicating a one-category change in MI measure 1. In the second definition, a responder was a patient who entered the study as “Very heavy” or “Heavy” (MI measure 1) and then, following treatment (month 1), indicated being “Moderate” or “Light”. When the treatment group was analyzed using the first responder definition, 69 (90%) of the 77 responders reported improvement and 63 (91%) of these rated this improvement as “a meaningful change”. Thirty-five (71%) of the 49 non-responders reported improvement and 35 (92%) rated their change as “a meaningful change”.

When the treatment group was analyzed using the second responder definition, 57 (89%) of the 64 responders reported improvement, and 52 (91%) reported their change to be meaningful. Forty-seven (76%) of the 62 non-responders reported improvement, and 45 (90%) reported their change to be meaningful. Among the normal group, 96 (73%) of 130 patients reported no change. Twenty-one (16%) reported improvement, and 13 (10%) reported worsening. Of the patients reporting change, 15 (44%) rated the change as “a meaningful change”.

For those women on treatment who reported a meaningful improvement (78.6%), MI items 3 and 4 showed the greatest treatment effect with improvements of 1.29 and 1.17, respectively. As expected, the majority of the Normal cohort (73.3%) reported no change in their menstrual period.

### Examples 17A-B

### Summary of Clinical Findings

The efficacy and safety of the tranexamic acid (TXA MR) modified release tablets in the treatment of HMB was demonstrated in one 3-cycle treatment and one 6-cycle treatment, randomized, double-blind, placebo-controlled study. In these studies, the primary outcome measure was menstrual blood loss (MBL), measured using a validated menstrual blood loss method. The key secondary outcome measures were based on responses to items on the Menorrhagia Instrument (MI), a validated disease-specific patient-reported outcome instrument that measured Limitations in Social or Leisure activities and Limitations in Physical Activities. Large stains (soiling beyond the undergarment) and sanitary product use were also included as secondary outcome measures. In these studies, subjects were 18 to 49 years of age with a mean age of approximately 40 years and a BMI of approximately 32 kg/m2. On average, subjects had an HMB history of approximately 10 years and 40% had fibroids as determined by transvaginal ultrasound. About 20% were smokers and approximately 50% reported using alcohol. Approximately 70% were Caucasian, 25% were Black, and 5% were Asian, Native American, Pacific Islander, or Other. Seven percent (7%) of subjects were of Hispanic origin. In addition, approximately 18% of subjects were taking multivitamins and 7% of subjects were taking iron supplements.

### Example 17A

### Three-Cycle Treatment Study

This study compared the effects of two doses of tranexamic acid modified release tablets (1.95 g and 3.9 g given daily for up to 5 days during each menstrual period) versus placebo on MBL over a 3-cycle treatment duration. A total of 304 patients (117 TXA MR 1.95 g/day, 118 TXA MR 3.9 g/day, 69 Placebo) were randomized. MBL was significantly reduced in patients treated with 3.9 g/day TXA MR compared to placebo (mean 3.9 g/day TXA MR=65.31 mL [percent MBL reduction=38.6%]; placebo mean=2.98 mL [percent MBL reduction=11.9%]; p<0.0001). This reduction met the criteria for being a clinically meaningful improvement (MBL≧50 mL) and a meaningful improvement to women who participated in the trial (MBL≧36 mL). The 1.95 g/day dose did not meet the clinically meaningful improvement criteria for efficacy thereby establishing 3.9 g/day TXA MR as the minimally effective dose.

Tranexamic acid modified release tablets also significantly reduced limitations on social, leisure, and physical activities as measured by questions on the MI, and sanitary products used in the 3.9 g/day dose group compared to placebo (see Table 19). No significant treatment differences were observed in response rates on the number of large stains.

TABLE 19
Secondary Outcomes in 3-Cycle Study

| | | Mean (SD) | P-value vs. |
|---|---|---|---|
| Outcome Measure | N | Reduction* | Placebo |
| Social and Leisure Activities (MI) | | | |
| 3.9 gm/day TXA MR | 112 | 1.10 (1.12) | <0.0001 |
| Placebo | 66 | 0.34 (0.85) | |
| Physical Activities (MI) | | | |
| 3.9 gm/day TXA MR | 112 | 0.97 (1.03) | <0.0001 |
| Placebo | 66 | 0.32 (0.80) | |
| Sanitary Products Used | | | |
| 3.9 gm/day TXA MR | 112 | 6.36 (6.80) | <0.0001 |
| Placebo | 67 | 2.40 (6.13) | |
| Reduction in Large Stains** | | | |
| 3.9 gm/day TXA MR | 111 | 71 (64.0) | 0.156 |
| Placebo | 67 | 35 (52.2) | |

*Positive means reflect a decrease from baseline
**The reduction in large stains is reported as the number (%) of women who were classified as responders (i.e., subjects who experienced a positive change from baseline)

### Example 17B

### Six-Cycle Treatment Study

This study compared die effects of one dose of TXA MR (3.9 g/day) versus placebo on MBL over a 6-cycle treatment duration. A total of 196 patients (123 TXA MR 3.9 g/day, 73 Placebo) were randomized. Replicating the results from the 3-cycle treatment study, MBL was significantly reduced in patients treated with 3.9 g/day TXA MR compared to placebo (mean 3.9 g/day TXA MR=69.6 mL [percent MBL reduction=40.4%]; placebo mean=12.6 mL [percent MBL reduction=8.2%]; p<0.0001). This reduction met the criterion for being a clinically meaningful improvement (MBL≧50 mL) and a meaningful improvement to women (MBL≧36 mL). Limitations on social, leisure, and physical activities were also significantly reduced in the 3.9 g/day TXA MR dose group compared to placebo (see Table 20). No significant treatment differences were observed in sanitary products used or in response rates on the number of large stains.

TABLE 20
Secondary Outcomes in 6-Cycle Study

| | | Mean (SD) | P-value vs. |
|---|---|---|---|
| Outcome Measure | N | Reduction* | Placebo |
| Social and Leisure Activities (MI) | | | |
| 3.9 gm/day TXA MR | 115 | 0.89 (0.85) | <0.0001 |
| Placebo | 72 | 0.38 (0.82) | |
| Physical Activities (MI) | | | |
| 3.9 gm/day TXA MR | 115 | 0.90 (0.86) | <0.0001 |
| Placebo | 72 | 0.35 (0.90) | |
| Sanitary Products Used | | | |
| 3.9 gm/day TXA MR | 115 | 5.20 (6.39) | 0.129 |
| Placebo | 72 | 4.03 (5.94) | |
| Reduction in Large Stains** | | | |
| 3.9 gm/day TXA MR | 115 | 66 (57.4) | 0.453 |
| Placebo | 72 | 37 (51.4) | |

*Positive means reflect a decrease from baseline
**The reduction in large stains is reported as the number (%) of women who were classified as responders (i.e., subjects who experienced a positive change from baseline)

Other variations or embodiments of the invention will also be apparent to one of ordinary skill in the art from the above descriptions and examples. Thus, the forgoing embodiments are not to be construed as limiting the scope of this invention.

What is claimed is:

1. A delayed release oral dosage form comprising:
a core comprising tranexamic acid or a pharmaceutically acceptable salt thereof and a pharmaceutically acceptable excipient; and
a delayed release material coated on the core which provides for the delayed release of the tranexamic acid or pharmaceutically acceptable salt thereof such that the dosage form is suitable for administration on a two or three times a day basis.

2. The delayed release oral dosage form of claim 1, wherein the dosage form comprises from about 585 to about 715 mg of tranexamic acid or pharmaceutically acceptable salt thereof.

3. The delayed release oral dosage form of claim 1, which provides for the reduction of at least one side effect selected from the group consisting of headache, nausea, or combination thereof, as compared to an equivalent amount of tranexamic acid or pharmaceutically acceptable salt thereof in an immediate release oral dosage form when administered across a patient population.

4. The delayed release oral dosage form of claim 1, which provides less than about 25 percent incidence of headache as a side effect after single dose oral administration across a patient population.

5. The delayed release oral dosage form of claim 1, which provides less than about 10 percent incidence of nausea as a side effect after single dose oral administration across a patient population.

6. The delayed release oral dosage form of claim 1, wherein said core is overcoated with a seal coating prior to the delayed release coating.

7. The delayed release oral dosage form of claim 1, further comprising a film-coating over said delayed release coating.

8. The delayed release oral dosage form of claim 1, which provides an in-vitro dissolution release rate of the tranexamic acid or pharmaceutically acceptable salt thereof when measured by the USP 27 Apparatus Type II Paddle Method @ 50 RPM and 37±0.5° C. of less than about 10% by weight tranexamic acid or pharmaceutically acceptable salt thereof released by about 120 minutes in acid medium (1000 ml of 0.1N hydrochloric acid), and at least about 75% by weight of said tranexamic acid or pharmaceutically acceptable salt thereof released by about 45 minutes after subsequent immersion in buffer medium (1000 ml of pH 6.8 phosphate buffer)

9. The delayed release oral dosage form of claim 1, which provide for the delayed release of the tranexamic acid or pharmaceutically acceptable salt thereof such that substantially none of said tranexamic acid or pharmaceutically acceptable salt thereof is released from said dosage form after exposure of said dosage form for a period of 2 hours to an environmental fluid having a pH of less than about 2, and substantially all of said tranexamic acid is released from said dosage form after exposure of said dosage form for a period of 1 hour to an environmental fluid having a pH of at least about 5.

10. A method of treating menorrhagia comprising administering two or three times a day at least one oral dosage form of claim 1 to a human patient suffering from menorrhagia.

11. A delayed release oral dosage form comprising:
a matrix core comprising tranexamic acid or a pharmaceutically acceptable salt thereof and a delayed release material which provides for the delayed release of the tranexamic acid or pharmaceutically acceptable salt thereof such that the dosage form is suitable for administration on a two or three times a day basis.

12. The delayed release oral dosage form of claim 11, wherein the dosage form comprises from about 585 to about 715 mg of tranexamic acid or pharmaceutically acceptable salt thereof.

13. The delayed release oral dosage form of claim 11, which provides for the reduction of at least one side effect selected from the group consisting of headache, nausea, or combination thereof, as compared to an equivalent amount of tranexamic acid or pharmaceutically acceptable salt thereof in an immediate release oral dosage form when administered across a patient population.

14. The delayed release oral dosage form of claim 11, which provides less than about 25 percent incidence of headache as a side effect after single dose oral administration across a patient population.

15. The delayed release oral dosage form of claim 11, which provides less than about 10 percent incidence of nausea as a side effect after single dose oral administration across a patient population.

16. The delayed release oral dosage form of claim 11, which provides an in-vitro dissolution release rate of the tranexamic acid or pharmaceutically acceptable salt thereof when measured by the USP 27 Apparatus Type II Paddle Method @ 50 RPM and 37±0.5° C. of less than about 10% by weight tranexamic acid or pharmaceutically acceptable salt thereof released by about 120 minutes in acid medium (1000 ml of 0.1N hydrochloric acid), and at least about 75% by weight of said tranexamic acid or pharmaceutically acceptable salt thereof released by about 45 minutes after subsequent immersion in buffer medium (1000 ml of pH 6.8 phosphate buffer)

17. The delayed release oral dosage form of claim 11, which provide for the delayed release of the tranexamic acid or pharmaceutically acceptable salt thereof such that substantially none of said tranexamic acid or pharmaceutically acceptable salt thereof is released from said dosage form after exposure of said dosage form for a period of 2 hours to an environmental fluid having a pH of less than about 2, and substantially all of said tranexamic acid is released from said dosage form after exposure of said dosage form for a period of 1 hour to an environmental fluid having a pH of at least about 5.

18. A method of treating menorrhagia comprising administering two or three times a day at least one oral dosage form of claim 11 to a human patient suffering from menorrhagia.

19. A delayed release oral dosage form comprising:
a plurality of multiparticulates comprising tranexamic acid or a pharmaceutically acceptable salt thereof, and a delayed release material which provides for the delayed release for the tranexamic acid or pharmaceutically acceptable salt thereof such that the dosage form is suitable for administration on a two or three times a day basis.

20. The delayed release oral dosage form of claim 19, wherein the plurality multiparticulates are plurality of inert beads coated with said tranexamic acid and a delayed release material.
